(19) United States
(12) Patent Application Publication
(10) Pub. No.: US 2023/0355803 A1
(43) Pub. Date: Nov. 9, 2023

(54) EXTRACELLULAR VESICLES WITH IMMUNE MODULATORS

(71) Applicant: Chameleon Biosciences, Inc., Berkeley, CA (US)

(72) Inventor: Genine WINSLOW, San Anselmo, CA (US)

(21) Appl. No.: 18/012,555

(22) PCT Filed: Jun. 23, 2021
(86) PCT No.: PCT/US2021/038739

§ 371 Date: Dec. 22, 2022

Related U.S. Application Data
(60) Provisional application No. 63/043,587, filed on Jun. 24, 2020.

(51) Int. Cl. A61K 48/00; C07K 14/705; C12N 15/86

(52) U.S. Cl. CPC A61K 48/0025; C07K 14/70521; C07K 14/70532; C12N 15/86; A61K 38/00

(57) ABSTRACT

Provided is herein are extracellular vesicles (EVs) comprising a lipid bilayer comprising one or more immunosuppressive molecules. Also provided herein are methods to induce tolerance to a therapeutic agent such as AAV, using the EVs described herein.

### CROSS-REFERENCE TO RELATED APPLICATIONS

This application claims the priority benefit of U.S. Provisional Application No. 63/043,587, filed Jun. 24, 2020, the entire disclosure of which is hereby incorporated by reference.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 774392000240SeqList.txt, date recorded: Jun. 22, 2021, size:21 KB).

### FIELD OF THE INVENTION

The present disclosure relates generally to extracellular vesicles (EVs) with immune modulators for inducing immune tolerance in an individual.

### BACKGROUND

Undesired immune responses contribute to anti-drug responses and transplant rejection. Immune responses to drugs, particularly biologics, cell therapies, and gene therapies, can impact efficacy and prevent readministration. Pathogenic immune responses after transplantation of a donor organ in a receiving organism can lead to rejection of the transplant and decreased patient survival. Therefore, approaches to establish immunological tolerance to antigens are a focus of intense therapeutic development.

In terms of gene therapies, host immune responses to viral vectors prevent administration of second doses of product primarily due to capsid specific adaptive immune responses. Additionally, T cell responses to novel expression of a therapeutic protein may reduce efficacy of gene therapy products (Mingozzi et al. (2013) Blood, 122(1):23-36).

There remains a need for methods to reduce anti-drug responses and transplant rejection.

Exosomes with immunomodulatory agents are described in WO 2019/133934, WO 2019/178113, WO 2021/003445, WO 2018/208670, WO 2019/027847, and WO 2020/257710, all are incorporated herein by reference in their entirety.

All references cited herein, including patent applications and publications, are incorporated herein by reference in their entirety.

### BRIEF SUMMARY OF THE INVENTION

In some aspects, the invention provides an extracellular vesicle (EV) for inducing immune tolerance to an agent in an individual, wherein EV comprises a lipid bilayer comprising one or more immunosuppressive molecules. In some embodiments, the one or more immunosuppressive molecules comprise one or more immune checkpoint proteins. In some embodiments, the one or more immunosuppressive molecules comprise one or more of CTLA4, B7-1, B7-2, PD-1, PD-L1, PD-L2, CD28, VISTA, TIM-3, GAL9, TIGIT, CD155, LAG3, VISTA, BTLA, or HVEM. In some embodiments, the one or more immunosuppressive molecules targets CD40 or CD40L. In some embodiments, the immunosuppressive molecule is an antibody that binds CD40 or CD40L. In some embodiments, the lipid bilayer comprises two or more, three or more, or four or more different immunosuppressive molecules; or comprises two or more, three or more, or four or more different checkpoint proteins. In some embodiments, the lipid bilayer comprises CTLA4 and PD-L1; CTLA and PD-L2; CTLA-4 and VISTA; CTLA-4 and TIM-3, PD-L1 and PD-L2; PD-L1 and VISTA; PD-L1 and TIM-3, PD-L2 and VISTA; CTLA4 and PD-L1 and PD-L2; CTLA4 and PD-L1 and VISTA; CTLA4 and PD-L1 and TIM-3; CTLA4 and PD-L2 and VISTA; CTLA4 and PD-L2 and TIM-3; PD-L1 and PD-L2 and VISTA; PD-L1 and PD-L2 and TIM-3; CTLA4 and PD-L1 and PD-L1 and VISTA; CTLA4 and PD-L1 and PD-L1 and TIM-3; or CTLA4 and PD-L1 and PD-L1 and VISTA and TIM-3. In some embodiments, one or more of the immunosuppressive molecules comprises a transmembrane domain. In some embodiments, the transmembrane domain is a PDGR transmembrane domain.

In further embodiments, the lipid bilayer of the EV of the invention further comprises a targeting molecule. In some embodiments, the targeting molecule confers cell-or tissue-specificity to the EV. In some embodiments, the targeting molecule confers specificity of the EV to the liver, spleen, and/or thymus. In some embodiments, the targeting molecule targets MHC class I or MHC class II mismatches between donor tissue and the individual. In some embodiments, the targeting molecule is an antibody. In some embodiments, the one or more targeting molecules comprises a transmembrane domain.

In further embodiments, the EV of the invention is produced from a producer cell engineered to express the one or more immunosuppressive molecules. In some embodiments, the EV is produced from a producer cell engineered to express the one or more immunosuppressive molecules. In some embodiments, the EV is produced from a producer cell engineered to express one or more of CTLA4, B7-1, B7-2, PD-1, PD-L1, PD-L2, CD28, VISTA, TIM-3, GAL9, TIGIT, CD155, LAG3, VISTA, BTLA. HVEM, an anti-CD40 antibody or an anti-CD40L antibody. In some embodiments, the producer cell is a human embryonic kidney 293 (HEK 293) cell, HeLa cell, or a Per.C6 cell. In some embodiments, the EV is produced from a producer cell which contains no additional heterologous molecules other than the one or more immunosuppressive molecules and targeting molecules. In some embodiments, the EV contains no additional molecules heterologous to the cell from which it was derived other than the one or more immunosuppressive molecules and targeting molecules.

In some embodiments, the invention provides a composition comprising the EV of the invention (e.g., as described above) and one or more pharmaceutically acceptable excipients. In some embodiments, the composition further comprises an agent. In some embodiments, the agent is a therapeutic agent. In some embodiments, the agent is a polypeptide, a nucleic acid, a polypeptide-nucleic acid complex, a viral vector, a liposome, or transplanted cells or tissue. In some embodiments, the agent associates with the EV. In some embodiments, the agent associates with the exterior surface of the EV.

In some aspects, the invention provides methods for inducing immune tolerance to an agent in an individual, the method comprising administering an effective amount of an EV to the individual in conjunction with administering the agent to the individual, wherein EV comprises a lipid bilayer comprising one or more immunosuppressive molecules. In some embodiments, the one or more immunosuppressive molecules comprise one or more immune checkpoint proteins. In some embodiments, the one or more immunosuppressive molecules comprise one or more of CTLA4, B7-1, B7-2, PD-1, PD-L1, PD-L2, CD28, VISTA, TIM-3, GAL9, TIGIT, CD155, LAG3, VISTA, BTLA or HVEM. In some embodiments, the one or more immunosuppressive molecules targets CD40 or CD40L. In some embodiments, the immunosuppressive molecule is an antibody that binds CD40 or CD40L. In some embodiments, the lipid bilayer comprises two or more, three or more, or four or more different immunosuppressive molecules; or comprises two or more, three or more, or four or more different checkpoint proteins. In some embodiments, the lipid bilayer comprises CTLA4 and PD-L1; CTLA and PD-L2; CTLA-4 and VISTA; CTLA-4 and TIM-3, PD-L1 and PD-L2; PD-L1 and VISTA; PD-L1 and TIM-3, PD-L2 and VISTA; CTLA4 and PD-L1 and PD-L2; CTLA4 and PD-L1 and VISTA; CTLA4 and PD-L1 and TIM-3; CTLA4 and PD-L2 and VISTA; CTLA4 and PD-L2 and TIM-3; PD-L1 and PD-L2 and VISTA; PD-L1 and PD-L2 and TIM-3; CTLA4 and PD-L1 and PD-L1 and VISTA; CTLA4 and PD-L1 and PD-L1 and TIM-3; or CTLA4 and PD-L1 and PD-L1 and VISTA and TIM-3. In some embodiments, one or more of the immunosuppressive molecules comprises a transmembrane domain. In some embodiments, the transmembrane domain is a PDGFR transmembrane domain, an EGFR transmembrane domain, or a murine CTLA4 transmembrane domain.

In further embodiments of the methods of the invention the lipid bilayer further comprises a targeting molecule. In some embodiments, the targeting molecule confers cell-or tissue-specificity to the EV. In some embodiments, the targeting molecule confers specificity of the method to the liver, spleen, and/or thymus. In some embodiments, the targeting molecule targets MHC class I or MHC class II mismatches between donor tissue and the individual. In some embodiments, the targeting molecule is an antibody. In some embodiments, the one or more targeting molecules comprises a transmembrane domain.

In some embodiments of the methods of the invention, the EV is produced from a producer cell engineered to express the one or more immunosuppressive molecules. In some embodiments, the EV is produced from a producer cell engineered to express the one or more immunosuppressive molecules. In some embodiments, the EV is produced from a producer cell engineered to express one or more of CTLA4, B7-1, B7-2, PD-1, PD-L1, PD-L2, CD28, VISTA, TIM-3, GAL9, TIGIT, CD155, LAG3, VISTA, BTLA, HVEM, an anti-CD40 antibody or an anti-CD40L antibody. In some embodiments, the producer cell is a human embryonic kidney 293 (HEK 293) cell, HeLa cell, or a Per.C6 cell. In some embodiments, the EV is produced from a producer cell which contains no additional heterologous molecules other than the one or more immunosuppressive molecules and targeting molecules. In some embodiments, the EV contains no additional molecules heterologous to the cell from which it was derived other than the one or more immunosuppressive molecules and targeting molecules.

In some embodiments of the methods of the invention, the EV is administered to the individual before, at the same time, or after administration of the agent. In some embodiments, the EV is administered to the individual at the same time as administration of the agent. In some embodiments, the EV and the agent are in different formulations. In some embodiments, the EV and the agent are in the same formulation. In some embodiments, the agent associates with the EV. In some embodiments, the agent associates with the exterior surface of the EV.

In some embodiments of the methods of the invention, the stimulation of immune tolerance facilitates repeat administration of the agent to the individual. In some embodiments, the repeat administration comprises more than about 2 administrations, 3 administrations, 4 administrations, 5 administrations, 6 administrations, 7 administrations, 8 administrations, 9 administrations, or 10 administrations of the agent.

In some embodiments of the methods of the invention, the agent is a therapeutic agent. In some embodiments, the agent is a polypeptide, a nucleic acid, a polypeptide-nucleic acid complex, a viral vector, a liposome, or transplanted cells or tissue. In some embodiments, the agent is a therapeutic polypeptide. In some embodiments, the therapeutic polypeptide is an enzyme, a hormone, an antibody, an antibody fragment, a clotting factor, a growth factor, a receptor, or a functional derivative thereof. In some embodiments, the therapeutic polypeptide is Factor VIII, Factor IX, myotubularin, survival motor neuron protein (SMN), retinoid isomerohydrolase (RPE65), NADH-ubiquinone oxidoreductase chain 4, Choroideremia protein (CHM), huntingtin, alpha-galactosidase A, acid beta-glucosidase, alpha-glucosidase, ornithine transcarbomylase, argininosuccinate synthetase, β-globin, γ-globin, phenylalanine hydroxylase, or adrenoleukodystrophy protein (ALD). In some embodiments, the agent is a nucleic acid encoding a therapeutic polypeptide or a therapeutic nucleic acid. In some embodiments, the nucleic acid encodes Factor VIII, Factor IX, myotubularin, survival motor neuron protein (SMN), retinoid isomerohydrolase (RPE65), NADH-ubiquinone oxidoreductase chain 4, Choroideremia protein (CHM), huntingtin, alpha-galactosidase A, acid beta-glucosidase, alpha-glucosidase, ornithine transcarbomylase, argininosuccinate synthetase, β-globin, γ-globin, phenylalanine hydroxylase, or adrenoleukodystrophy protein (ALD). In some embodiments, the therapeutic nucleic acid is a siRNA, miRNA, shRNA, antisense RNA, RNAzyme, or DNAzyme. In some embodiments, the nucleic acid encodes one or more gene editing products. In some embodiments, the polypeptide-nucleic acid complex is a gene editing complex. In some embodiments, the agent is a viral vector or a capsid protein thereof. In some embodiments, the viral vector is an adeno-associated viral (AAV) vector, a lentiviral vector, an adenoviral vector, a herpes simplex viral vector or a baculovirus vector. In some embodiments of the methods of the invention, the individual is a human.

In some embodiments of the invention, the agent is a cell used in cell therapy. In some embodiments, the cell is a stem cell, an induced pluripotent cell (iPS), or a differentiated cell. In some embodiments, the cell is a pluripotent cell or a multipotent cell. In some embodiments, the cell is an embryonic stem cell or an adult stem cell. In some embodiments, the cell is a hematopoietic stem cell, a liver stem cell, a muscle stem cell, a cardiomyocyte stem cell, a neural stem cell, a bone stem cell, a mesenchymal stem cell, or an adipose stem cell. In some embodiments, the cell is a blood cell, a hepatocyte, a myocyte, a cardiomyocyte, a pancreatic cell, an islet cell, an ocular cell, a neural cell, an astrocyte, an oligodendrocyte, an inner ear hair cell, a chondrocyte, or an osteoblast. In some embodiments, the cell is allogeneic to the individual.

In some aspects, the invention provides methods for treating a disease or disorder in an individual, the method comprising administering an effective amount of an EV to the individual, wherein EV comprises a lipid bilayer comprising one or more immunosuppressive molecules. In some embodiments, the disease or disorder is an autoimmune disease or disorder. In some embodiments, the EV is administered in conjunction with a tissue transplant or cell engraftment.

In some aspects, the invention provides methods for treating a disease or disorder in an individual, the method comprising administering an effective amount of an EV to the individual in conjunction with administering an agent to the individual, wherein EV comprises a lipid bilayer comprising one or more immunosuppressive molecules, and wherein the agent treats the disease or disorder. In some embodiments, the one or more immunosuppressive molecules comprise one or more immune checkpoint proteins. In some embodiments, the one or more immunosuppressive molecules comprise one or more of CTLA4, B7-1, B7-2, PD-1, PD-L1, PD-L2, CD28, VISTA, TIM-3, GAL9, TIGIT, CD155, LAG3, VISTA, BTLA or HVEM. In some embodiments, the one or more immunosuppressive molecules targets CD40 or CD40L. In some embodiments, the immunosuppressive molecule is an antibody that binds CD40 or CD40L. In some embodiments, the lipid bilayer comprises two or more, three or more, or four or more different immunosuppressive molecules; or comprises two or more, three or more, or four or more different checkpoint proteins. In some embodiments, the lipid bilayer comprises CTLA4 and PD-L1; CTLA and PD-L2; CTLA-4 and VISTA; PD-L1 and PD-L2; PD-L1 and VISTA; PD-L2 and VISTA; CTLA4 and PD-L1 and PD-L2; CTLA4 and PD-L1 and VISTA; CTLA4 and PD-L2 and VISTA; PD-L1 and PD-L2 and VISTA; or CTLA4 and PD-L1 and PD-L1 and VISTA. In some embodiments, one or more of the immunosuppressive molecules comprises a transmembrane domain. In some embodiments, the transmembrane domain is a PDGFR transmembrane domain, an EGFR transmembrane domain, or a murine CTLA4 transmembrane domain.

In some embodiments of the methods of treatment of the invention, the lipid bilayer further comprises a targeting molecule. In some embodiments, the targeting molecule confers cell- or tissue-specificity to the EV. In some embodiments, the targeting molecule confers specificity of the method to the liver, spleen, and/or thymus. In some embodiments, the targeting molecule targets MHC class I or MHC class II mismatches between donor tissue and the individual. In some embodiments, the targeting molecule is an antibody. In some embodiments, the one or more targeting molecules comprises a transmembrane domain.

In some embodiments of the methods of treatment of the invention, the EV is produced from a producer cell engineered to express the one or more immunosuppressive molecules. In some embodiments, the EV is produced from a producer cell engineered to express the one or more immunosuppressive molecules. In some embodiments, the EV is produced from a producer cell engineered to express one or more of CTLA4, B7-1, B7-2, PD-1, PD-L1, PD-L2, CD28, VISTA, TIM-3, GAL9, TIGIT, CD155, LAG3, VISTA, BTLA, HVEM, an anti-CD40 antibody or an anti-CD40L antibody. In some embodiments, the producer cell is a human embryonic kidney 293 (HEK 293) cell, HeLa cell, or a Per.C6 cell. In some embodiments, the EV is produced from a producer cell which contains no additional heterologous molecules other than the one or more immunosuppressive molecules and targeting molecules. In some embodiments, the EV contains no additional molecules heterologous to the cell from which it was derived other than the one or more immunosuppressive molecules and targeting molecules.

In some embodiments of the methods of treatment of the invention, the EV is administered to the individual before, at the same time, or after administration of the agent. In some embodiments, the EV is administered to the individual at the same time as administration of the agent. In some embodiments, the EV and the agent are in different formulations. In some embodiments, the EV and the agent are in the same formulation. In some embodiments, the agent associates with the EV. In some embodiments, the agent associates with the exterior surface of the EV. In some embodiments, the stimulation of immune tolerance facilitates repeat administration of the agent to the individual. In some embodiments, the repeat administration comprises more than about 2 administrations, 3 administrations, 4 administrations, 5 administrations, 6 administrations, 7 administrations, 8 administrations, 9 administrations, or 10 administrations of the agent.

In some embodiments of the methods of treatment of the invention, the agent is a therapeutic agent. In some embodiments, the agent is a polypeptide, a nucleic acid, a polypeptide-nucleic acid complex, a viral vector, a liposome, or transplanted cells or tissue. In some embodiments, the agent is a therapeutic polypeptide. In some embodiments, the therapeutic polypeptide is an enzyme, a hormone, an antibody, an antibody fragment, a clotting factor, a growth factor, a receptor, or a functional derivative thereof. In some embodiments, the therapeutic polypeptide is Factor VIII, Factor IX, myotubularin, survival motor neuron protein (SMN), retinoid isomerohydrolase (RPE65), NADH-ubiquinone oxidoreductase chain 4, Choroideremia protein (CHM), huntingtin, alpha-galactosidase A, acid beta-glucosidase, alpha-glucosidase, ornithine transcarbomylase, argininosuccinate synthetase, β-globin, γ-globin, phenylalanine hydroxylase, or adrenoleukodystrophy protein (ALD). In some embodiments, the agent is a nucleic acid encoding a therapeutic polypeptide or a therapeutic nucleic acid. In some embodiments, the nucleic acid encodes Factor VIII, Factor IX, myotubularin, survival motor neuron protein (SMN), retinoid isomerohydrolase (RPE65), NADH-ubiquinone oxidoreductase chain 4, Choroideremia protein (CHM), huntingtin, alpha-galactosidase A, acid beta-glucosidase, alpha-glucosidase, ornithine transcarbomylase, argininosuccinate synthetase, β-globin, γ-globin, phenylalanine hydroxylase, or adrenoleukodystrophy protein (ALD). In some embodiments, the therapeutic nucleic acid is a siRNA, miRNA, shRNA, antisense RNA, RNAzyme, or DNAzyme. In some embodiments, the nucleic acid encodes one or more gene editing products. In some embodiments, the polypeptide-nucleic acid complex is a gene editing complex. In some embodiments, the agent is a viral vector or a capsid protein thereof. In some embodiments, the viral vector is an adeno-associated viral (AAV) vector, a lentiviral vector, an adenoviral vector, a herpes simplex viral vector or a baculovirus. In some embodiments, the individual is a human.

In some aspects, the invention provide methods for producing an EV of the invention, the method comprising culturing EV producer cells in vitro under conditions to generate EVs, wherein the EV producer cells comprise nucleic acids encoding one or more one or more membrane-bound immunosuppressive molecules, and collecting the EVs. In some embodiments, the EV producer cells comprise exogenous nucleic acids encoding the membrane-bound immunosuppressive molecules. In some embodiments, the membrane-bound immunosuppressive molecules comprise one or more of CTLA4, B7-1, B7-2, PD-1, PD-L1, PD-L2, CD28, VISTA, TIM-3, GAL9, TIGIT, CD155, LAG3, VISTA, BTLA or HVEM. In some embodiments, the one or more immunosuppressive molecules targets CD40 or CD40L. In some embodiments, the immunosuppressive molecule is an antibody that binds CD40 or CD40L. In some embodiments, one or more of the immunosuppressive molecules comprises a transmembrane domain. In some embodiments, the transmembrane domain is a PDGFR transmembrane domain, an EGFR transmembrane domain, or a murine CTLA4 transmembrane domain.

In some embodiments of the methods of producing an EV of the invention, the lipid bilayer further comprises a targeting molecule. In some embodiments, the targeting molecule confers cell- or tissue-specificity to the EV. In some embodiments, the targeting molecule confers specificity of the EV to the liver, spleen, and/or thymus. In some embodiments, the targeting molecule targets MHC class I or MHC class II mismatches between donor tissue and the individual. In some embodiments, the targeting molecule is an antibody. In some embodiments, the one or more targeting molecules comprises a transmembrane domain.

In some embodiments, the EV is produced from a producer cell engineered to express the one or more immunosuppressive molecules. In some embodiments, the EV is produced from a producer cell engineered to express the one or more immunosuppressive molecules. In some embodiments, the EV is produced from a producer cell engineered to express one or more of CTLA4, B7-1, B7-2, PD-1, PD-L1, PD-L2, CD28, VISTA, TIM-3, GAL9, TIGIT, CD155, LAG3, VISTA, BTLA or HVEM. In some embodiments, the producer cell is a human embryonic kidney 293 (HEK 293) cell, HeLa cell, or a Per.C6 cell. In some embodiments, the EV is produced from a producer cell which contains no additional heterologous molecules other than the one or more immunosuppressive molecules and targeting molecules. In some embodiments, the EV contains no additional molecules heterologous to the cell from which it was derived other than the one or more immunosuppressive molecules and targeting molecules.

In some embodiments, the invention provides a producer cell for producing an immunosuppressive EV, wherein EV comprises a lipid bilayer comprising one or more immunosuppressive molecules, wherein the one or more immunosuppressive molecules are membrane-bound. In some embodiments, the producer cell is engineered to express the one or more immunosuppressive molecules. In some embodiments, the producer cell is engineered to express one or more of CTLA4, B7-1, B7-2, PD-1, PD-L1, PD-L2, CD28, VISTA, TIM-3, GAL9, TIGIT, CD155, LAG3, VISTA, BTLA or HVEM. In some embodiments, the one or more immunosuppressive molecules targets CD40 or CD40L. In some embodiments, the immunosuppressive molecule is an antibody that binds CD40 or CD40L. In some embodiments, one or more of the immunosuppressive molecules comprises a transmembrane domain. In some embodiments, the transmembrane domain is a PDGFR transmembrane domain, an EGFR transmembrane domain, or a murine CTLA4 transmembrane domain.

In some embodiments of the producer cells of the invention, the lipid bilayer of further comprises a targeting molecule. In some embodiments, the targeting molecule confers cell- or tissue-specificity to the EV. In some embodiments, the targeting molecule confers specificity of the EV to the liver, spleen, and/or thymus. In some embodiments, the targeting molecule targets MHC class I or MHC class II mismatches between donor tissue and the individual. In some embodiments, the targeting molecule is an antibody. In some embodiments, the one or more targeting molecules comprises a transmembrane domain.

In some embodiments, the producer cell of the invention comprises nucleic acid encoding the one or more immunosuppressive molecule and/or the one or more targeting molecule. In some embodiments, the nucleic acid encoding the one or more immunosuppressive molecule and/or the one or more targeting molecule is stably integrated into the genome of the cell.

In some embodiments of the invention, the producer cell is a mammalian cell. In some embodiments, the producer cell is a human cell. In some embodiments, the producer cell is a human embryonic kidney 293 (HEK 293) cell, HeLa cell, or a Per.C6 cell. In some embodiments, the producer cell contains no additional heterologous molecules other than the one or more immunosuppressive molecules and targeting molecules.

### BRIEF DESCRIPTION OF THE DRAWING

FIG. 1 shows an example of the functional components of an immune tolerizing extracellular vesicle with the transmembrane domain proteins CTLA-4 and PD-L1 incorporated into the lipid bilayer.

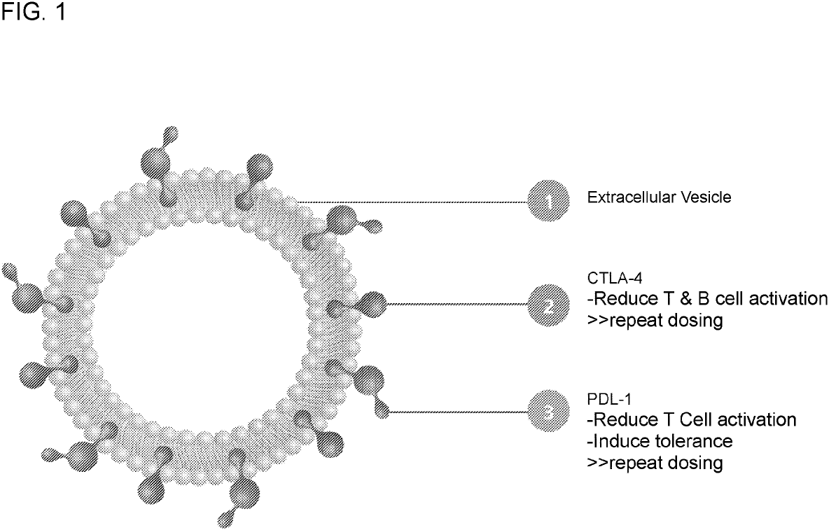

### DETAILED DESCRIPTION

In some aspects, the invention provides an extracellular vesicle (EV) for inducing immune tolerance to an agent in an individual, wherein EV comprises a lipid bilayer comprising one or more immunosuppressive molecules. In some embodiments, the one or more immunosuppressive molecules comprise one or more immune checkpoint proteins.

In some aspects, the invention provides methods of inducing immune tolerance to an agent in an individual, the method comprising administering an effective amount of an EV to the individual in conjunction with administering the agent to the individual, wherein EV comprises a lipid bilayer comprising one or more immunosuppressive molecules. In some embodiments, the one or more immunosuppressive molecules comprise one or more immune checkpoint proteins.

In some aspects, the invention provides methods of treating a disease or disorder in an individual, the method comprising administering an effective amount of an EV to the individual in conjunction with administering the agent to the individual, wherein EV comprises a lipid bilayer comprising one or more immunosuppressive molecules, and wherein the agent treats the disease or disorder. In some embodiments, the one or more immunosuppressive molecules comprise one or more immune checkpoint proteins.

In some aspects, the invention provides methods of producing an EV for inducing immune tolerance to an agent in an individual, wherein EV comprises a lipid bilayer comprising one or more immunosuppressive molecules, the method comprising culturing EV producer cells in vitro under conditions to generate EVs, wherein the EV producer cells comprise nucleic acids encoding one or more one or more membrane-bound immunosuppressive molecules, and collecting the EVs.

In some aspects, the invention provides producer cells for producing an immunosuppressive EV, wherein EV comprises a lipid bilayer comprising one or more immunosuppressive molecules, wherein the one or more immunosuppressive molecules are membrane-bound.

In some aspects, the invention provides a tolerizing extracellular vesicle that can be formulated with an agent (e.g., a therapeutic product), including but not limited to, recombinant antibodies, proteins, nucleic acids, cells, or virus capsids (e.g., engineered or wild type virus capsids); to induce immune tolerance to the agent.

In some aspects, the invention provides a tolerizing EV produced by the same producer cells that simultaneously produce a secreted agent that becomes associated with the EV in the producer cell supernatant. In some embodiments, the agent associates with the exterior surface of the EV.

### General Techniques

The techniques and procedures described or referenced herein are generally well understood and commonly employed using conventional methodology by those skilled in the art, such as, for example, the widely utilized methodologies described in Molecular Cloning: A Laboratory Manual (Sambrook et al., 4^{th} ed., Cold Spring Harbor Laboratory Press, Cold Spring Harbor, N.Y., 2012); Current Protocols in Molecular Biology (F.M. Ausubel, et al. eds., 2003); the series Methods in Enzymology (Academic Press, Inc.); PCR 2: A Practical Approach (M.J. MacPherson, B.D. Hames and G.R. Taylor eds., 1995); Antibodies, A Laboratory Manual (Harlow and Lane, eds., 1988); Culture of Animal Cells: A Manual of Basic Technique and Specialized Applications (R.I. Freshney, 6^{th} ed., J. Wiley and Sons, 2010); Oligonucleotide Synthesis (M.J. Gait, ed., 1984); Methods in Molecular Biology, Humana Press; Cell Biology: A Laboratory Notebook (J.E. Cellis, ed., Academic Press, 1998); Introduction to Cell and Tissue Culture (J.P. Mather and P.E. Roberts, Plenum Press, 1998); Cell and Tissue Culture: Laboratory Procedures (A. Doyle, J.B. Griffiths, and D.G. Newell, eds., J. Wiley and Sons, 1993-8); Handbook of Experimental Immunology (D.M. Weir and C.C. Blackwell, eds., 1996); Gene Transfer Vectors for Mammalian Cells (J.M. Miller and M.P. Calos, eds., 1987); PCR: The Polymerase Chain Reaction, (Mullis et al., eds., 1994); Current Protocols in Immunology (J.E. Coligan et al., eds., 1991); Short Protocols in Molecular Biology (Ausubel et al., eds., J. Wiley and Sons, 2002); Immunobiology (C.A. Janeway et al., 2004); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane, Cold Spring Harbor Laboratory Press, 1999); The Antibodies (M. Zanetti and J. D. Capra, eds., Harwood Academic Publishers, 1995); and Cancer: Principles and Practice of Oncology (V.T. DeVita et al., eds., J.B. Lippincott Company, 2011).

### Definitions

For purposes of interpreting this specification, the following definitions will apply unless otherwise stated. Whenever appropriate, terms used in the singular will also include the plural and vice versa. In the event that any definition set forth below conflicts with any document incorporated herein by reference, the definition set forth shall control.

As used herein, the singular form “a”, “an”, and “the” includes plural references unless indicated otherwise.

The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context.

It is understood that aspects and embodiments of the disclosure described herein include “comprising,” “consisting,” and “consisting essentially of” such aspects and embodiments.

For all compositions described herein, and all methods using a composition described herein, the compositions can either comprise the listed components or steps, or can “consist essentially of” or “consist of” the listed components or steps. When a composition is described as “consisting essentially of” the listed components, the composition contains the components listed, and may contain other components which do not substantially affect the methods disclosed, but do not contain any other components which substantially affect the methods disclosed other than those components expressly listed; or, if the composition does contain extra components other than those listed which substantially affect the methods disclosed, the composition does not contain a sufficient concentration or amount of the extra components to substantially affect the methods disclosed. When a method is described as “consisting essentially of” the listed steps, the method contains the steps listed, and may contain other steps that do not substantially affect the methods disclosed, but the method does not contain any other steps which substantially affect the methods disclosed other than those steps expressly listed. As a non-limiting specific example, when a composition is described as consisting essentially of′ a component, the composition may additionally contain any amount of pharmaceutically acceptable carriers, vehicles, or diluents and other such components which do not substantially affect the properties of composition or the methods disclosed.

The term “about” as used herein refers to the usual error range for the respective value readily known to the skilled person in this technical field. Reference to “about” a value or parameter herein includes (and describes) embodiments that are directed to that value or parameter per se.

As used herein, the term “extracellular vesicles” refers to a heterogeneous group of cell-derived membranous structures including EVs and microvesicles, which originate from the endosomal system or which are shed from the plasma membrane, respectively. For example, see van Niel, G. et al., Nat Rev Mol Cell Biol. 2018 Apr;19(4):213-228.

The term “polynucleotide” or “nucleic acid” as used herein refers to a polymeric form of nucleotides of any length, ribonucleotides, deoxyribonucleotides or combination therein. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be an oligodeoxynucleoside phosphoramidate (P-NH2) or a mixed phosphoramidate-phosphodiester oligomer. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The terms “polypeptide” and “protein” are used interchangeably to refer to a polymer of amino acid residues, and are not limited to any particular minimum or maximum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

A “viral vector” refers to a polynucleotide vector comprising one or more heterologous sequences (i.e., nucleic acid sequence not of viral origin) that are flanked by at least one or two repeat sequences (e.g., inverted terminal repeat sequences (ITRs) for AAV or long terminal repeats (LTRs) for lentivirus). The heterologous nucleic acid and be referred to as a “payload” to be delivered as a “cassette” and is often flanked by the at least one or two repeat sequences (e.g., inverted terminal repeat sequences (ITRs) for AAV or long terminal repeats (LTRs) for lentivirus). Such viral vectors can be replicated and packaged into infectious viral particles when present in a host cell provided that the host cell provides the essential functions. When a viral vector is incorporated into a larger polynucleotide (e.g., in a chromosome or in another vector such as a plasmid used for cloning or transfection), then the viral vector may be referred to as a “pro-vector” which can be “rescued” by replication and encapsidation in the presence of viral replication and packaging functions. A viral vector can be packaged into a virus capsid to generate a “viral particle”. In some respects, a viral particle refers to a virus capsid together with the viral genome and heterologous nucleic acid payload.

“Heterologous” means derived from a genotypically distinct entity from that of the rest of the entity to which it is compared or into which it is introduced or incorporated. For example, a polynucleotide introduced by genetic engineering techniques into a different cell type is a heterologous polynucleotide (and, when expressed, can encode a heterologous polypeptide). Similarly, a cellular sequence (e.g., a gene or portion thereof) that is incorporated into a viral vector is a heterologous nucleotide sequence with respect to the vector. A heterologous nucleic acid may refer to a nucleic acid derived from a genotypically distinct entity from that of the rest of the entity to which it is compared or into which it is introduced or incorporated. Heterologous also can be used to refer to other biological components (e.g., proteins) that are non-native to the species into which they are introduced. For instance, a protein expressed in a cell from a heterologous nucleic acid would be a heterologous protein with respect to the cell. A nucleic acid introduced into a cell or organism by genetic engineering techniques may be considered “exogenous” to the cell or organism regardless of whether it is heterologous or homologous to the cell or organism. Thus, for instance, a vector could be used to introduce an additional copy of human gene into a human cell. The gene introduced to the cell would be exogenous to the cell even though it might contain a homologous (native) nucleic acid sequence.

An “isolated” molecule (e.g., nucleic acid or protein) or cell means it has been identified and separated and/or recovered from a component of its natural environment.

“Engineered” or “genetically engineered” and like terms are used to refer to biological materials that are artificially genetically modified (e.g., using laboratory techniques) or result from such genetic modifications.

As used herein, “treatment” is an approach for obtaining beneficial or desired clinical results. For purposes of this invention, beneficial or desired clinical results include, but are not limited to, alleviation of symptoms, diminishment of extent of disease, stabilized (e.g., not worsening) state of disease, preventing spread (e.g., metastasis) of disease, delay or slowing of disease progression, amelioration or palliation of the disease state, and remission (whether partial or total), whether detectable or undetectable. “Treatment” can also mean prolonging survival as compared to expected survival if not receiving treatment.

As used herein, the term “prophylactic treatment” refers to treatment, wherein an individual is known or suspected to have or be at risk for having a disorder but has displayed no symptoms or minimal symptoms of the disorder. An individual undergoing prophylactic treatment may be treated prior to onset of symptoms.

An “effective amount” is an amount sufficient to effect beneficial or desired results, including clinical results (e.g., amelioration of symptoms, achievement of clinical endpoints, and the like). An effective amount can be administered in one or more administrations. In terms of a disease state, an effective amount is an amount sufficient to ameliorate, stabilize, or delay development of a disease.

As used herein, by “combination therapy” is meant that a first agent be administered in conjunction with another agent. “In conjunction with” refers to administration of one treatment modality in addition to another treatment modality, such as administration of a composition of EVs as described herein in addition to administration of an agent (e.g., a therapeutic agent) as described herein to the same individual. As such, “in conjunction with” refers to administration of one treatment modality before, during, or after delivery of the other treatment modality to the individual.

The term “simultaneous administration,” as used herein, means that a first therapy and second therapy in a combination therapy are administered with a time separation of no more than about 15 minutes, such as no more than about any of 10, 5, or 1 minutes. When the first and second therapies are administered simultaneously, the first and second therapies may be contained in the same composition (e.g., a composition comprising both a first and second therapy) or in separate compositions (e.g., a first therapy in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60, or more minutes. Either the first therapy or the second therapy may be administered first. The first and second therapies are contained in separate compositions, which may be contained in the same or different packages or kits.

As used herein, the term “concurrent administration” means that the administration of the first therapy and that of a second therapy in a combination therapy overlap with each other.

An “individual” or “subject” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). Particularly, the individual or subject is a human.

The term “pharmaceutical composition” refers to a preparation which is in such form as to permit the biological activity of an active ingredient contained therein to be effective, and which contains no additional components which are unacceptably toxic to a subject to which the composition would be administered.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

For any of the structural and functional characteristics described herein, methods of determining these characteristics are known in the art.

### EVs Engineered With Immunosuppressive Molecules

The EVs provided herein comprises a lipid bilayer comprising one or more immunomodulatory molecules (e.g., immunosuppressive molecules or immunostimulatory molecules). Any lipid bilayer can be used, including naturally occurring or synthetic (artificial) lipid bilayers. Synthetic lipid bilayers include, for example, liposomes. Naturally occurring lipid bilayers include any of various types of extracellular vesicles (EVs) known in the art, including exosomes, microvesicles (e.g., shedding vesicles or ectosomes), and the like. For instance, the lipid bilayer of the lipid bilayer of the EV can be provided by a portion of a cell membrane that has “budded” from a producer cell, particularly a producer cell that has been engineered to overexpress one or more immunosuppressive molecules as compared to a non-engineered producer cell of the same type. Such a lipid bilayer comprises a portion of a cell membrane from which it is shed. In some embodiments, the lipid bilayer comprises endosome-associated proteins (Alix, Tsg101, and Rab proteins); tetraspanins (CD9, CD63, CD81, CD82, CD53, and CD37); lipid raft-associated proteins (glycosylphosphatidylinositol and flotillin), and/or lipids comprising cholesterol, sphingomyelin, and/or glycerophospholipids. In some embodiments, the lipid bilayer is an exosomal lipid bilayer (e.g., the lipid bilayer is an exosome), particularly the exosomal lipid bilayer of a producer cell (i.e., shed from other otherwise derived from or produced by a producer cell) that is engineered to overexpress one or more immunosuppressive molecules as described herein.

While any cell type can provide EVs, it is sometimes advantageous to avoid the use of tumor cells as producer cells in the context of the invention, due to the potential for contamination by agents (e.g., genetic elements) that contribute to the immortalization of the tumor cell and might be oncogenic or otherwise detrimental to a subject. Thus, in some embodiments, the lipid bilayer is a non-tumor EV lipid bilayer, such as a non-tumor exosomal lipid bilayer (e.g., the lipid bilayer is from a non-tumor EV such as a non-tumor exosome, meaning that the EV or exosome does not have a tumor-cell origin). In other embodiments, the lipid bilayer is an EV lipid bilayer (e.g., an exosomal lipid bilayer or an exosome) from a 293 cell (e.g., HEK293 or HEK293T), particularly an EV lipid bilayer (e.g., an exosomal lipid bilayer or an exosome) a non-tumor producer cell (i.e., shed from other otherwise derived from or produced by a producer cell), such as a 293 cell, that is engineered to express (e.g., overexpress) one or more immunosuppressive molecules as described herein.

The lipid bilayer also comprises immunomodulatory molecules (e.g., immunosuppressive molecules or immunostimulatory molecules). In some embodiments, the lipid bilayer comprises immunosuppressive molecules. The immunosuppressive molecules can be associated with the lipid bilayer in any manner. In some embodiments, the immunosuppressive molecule is embedded within or on the lipid bilayer. For instance, the immunosuppressive molecule can comprise, either naturally or synthetically, a transmembrane domain, which integrates into the lipid bilayer. In some embodiments, the transmembrane domain is embedded in the lipid bilayer and at least a portion (e.g., a functional portion) of the immunosuppressive molecule is displayed on the exterior of the EV. In some embodiments, the transmembrane domain spans the lipid bilayer and at least a portion (e.g., a functional portion) of the immunosuppressive molecule is displayed on the exterior of the EV. Transmembrane domains are known in the art including but not limited to the PDGFR transmembrane domain, the EGFR transmembrane domain, or the murine CTLA4 transmembrane domain. In some embodiments, the transmembrane domain is any domain that efficiently traffics the immunosuppressive molecule and/or a targeting molecule to the plasma membrane of the producer cell. Methods of incorporating transmembrane domains (e.g., by generating fusion proteins) are known in the art.

The immunosuppressive molecule can be any molecule that reduces the host immune response to a therapeutic agent as compared to the same agent without coadiministering of the EV or with an EV that is not engineered to contain immunosuppressive molecules. The immunosuppressive molecules include but are not limited to molecules (e.g., proteins) that down-regulate immune function of a host by any mechanism, such as by stimulating or up-regulating immune inhibitors or by inhibiting or down-regulating immune stimulating molecules and/or activators. Immunosuppressive molecules include, but are not limited immune checkpoint receptors and ligands. Non-limiting examples of immunosuppressive molecules include, for instance, CTLA-4 and its ligands (e.g., B7-1 and B7-2), PD-1 and its ligands (e.g., PDL-1 and PDL-2), VISTA, TIM-3 and its ligand (e.g., GAL9), TIGIT and its ligand (e.g., CD155), LAG3, VISTA, and BTLA and its ligand (e.g., HVEM). Also included are active fragments and derivatives of any of the foregoing checkpoint molecules; agonists of any of the foregoing checkpoint molecules, such as agonistic antibodies to any of the foregoing checkpoint molecules; antibodies that block immune stimulatory receptors (co-stimulatory receptors) or their ligands, such as anti-CD28 antibodies; or peptides that mimic the immune functions of immune checkpoint molecules. To the extent a desired immunosuppressive molecule does not natively include a transmembrane domain, the immunosuppressive molecules can be engineered to embed in a lipid bilayer by creating chimeric molecules comprising an extracellular domain, a transmembrane domain, and, optionally, either full length intracellular domains, or any minimal intercellular domain that may be necessary to maintain chimeric molecule expression and binding to its ligand or receptor. The transmembrane domains and intercellular domains of effector molecules can comprise immunoglobulin Fc receptor domains (or transmembrane region thereof) or any other functional domain necessary to maintain expression and ligand binding activities.

In some embodiments, the immunosuppressive molecule inhibits the function of B cells. In some embodiments, the immunosuppressive molecule is an antagonist of CD40 or its ligand, CD40L (also known as CD154). In some embodiments, the immunosuppressive molecule is an antibody that specifically binds CD40 or its ligand, CD40L (also known as CD154).

The lipid bilayer can comprise any one or more different types of immunosuppressive molecules; however, in some embodiments, the lipid bilayer comprises a combination of two or more different immunosuppressive molecules (e.g., three or more different immunosuppressive molecules, four or more different immunosuppressive molecules, or even five or more different immunosuppressive molecules). Thus, for example, in some embodiments, the lipid bilayer comprises a combination of two or more different immune checkpoint molecules (e.g., three or more different immune checkpoint molecules, four or more different immune checkpoint molecules, or even five or more different immune checkpoint molecules), optionally two or more (e.g., three or more, four or more, or even five or more) molecules selected from CTLA-4 and its ligands (e.g., B7-1 and B7-2), PD-1 and its ligands (e.g., PDL-1 and PDL-2), VISTA, TIM-3 and its ligand (e.g., GAL9), TIGIT and its ligand (e.g., CD155), LAG3, VISTA, and BTLA and its ligand (e.g., HVEM); active fragments and derivatives of any of the foregoing checkpoint molecules; agonists of any of the foregoing checkpoint molecules, such as agonistic antibodies to any of the foregoing checkpoint molecules; antibodies that block immune stimulatory receptors (co-stimulatory receptors) or their ligands, such as anti-CD28 antibodies; or peptides that mimic the immune functions of immune checkpoint molecules. In some embodiments, the lipid bilayer comprises CTLA-4 and PD-L1 and PD-L2 and VISTA, or any combination of these, or other immune suppressing molecules, singly or in combinations of up to four different molecules. In some embodiments, the lipid bilayer comprises CTLA-4 and PD-L1, CTLA-4 and PD-L2, CTLA-4 and PD-1, CTLA-4 and VISTA, CTLA-4 and anti-CD28, PD-1 and VISTA, B7-1 and PD-L1, B7-1 and PD-L2, B7-1and PD-1, B7-1 and VISTA, B7-1 and anti-CD28, B7-2 and PD-L1, B7-2 and PD-L2, B7-2and PD-1, B7-2 and VISTA, B7-2 and anti-CD28, PD-1 and VISTA, PD-1 and anti-CD-28, VISTA and anti-CD28, PD-L1 and VISTA, PD-L1 and anti-CD-28, PD-L2 and VISTA, PD-L2 and anti-CD-28, or VISTA and anti-CD28. In some embodiments, the lipid bilayer comprises CTLA4 and PD-L1, CTLA and PD-L2 CTLA-4 and VISTA, PD-L1 and PD-L2, PD-L1 and VISTA, PD-L2 and VISTA, CTLA4 and PD-L1 and PD-L2, CTLA4 and PD-L1 and VISTA, CTLA4 and PD-L2 and VISTA, PD-L1 and PD-L2 and VISTA, or CTLA4 and PD-L1 and PD-L1 and VISTA.

In some embodiments, the immunosuppressive molecules are engineered to include a transmembrane domain. The immunosuppressive molecule used in the vector should be that of the species of mammal to which the vector is to be administered. Thus, for use in humans, the human ortholog of the immunosuppressive molecule should be used, which proteins are well-known in the field. In a particular embodiment, the immunosuppressive molecules included in the lipid bilayer comprise, consist essentially of, or consist of, CTLA-4 and PD-L1. Human CTLA-4 is provided, for instance, by the protein identified by NCBI Reference Sequence: NP_005205.2, and PD-L1 is provided, for instance, by the protein identified by NCBI Reference Sequence: NP_054862.1. In some embodiments, the immunosuppressive molecule is (or derived from) a CTLA-4 molecule comprising the amino acid sequence of SEQ ID NO:1. In some embodiments, the immunosuppressive molecule is (or derived from) a CTLA-4 molecule comprising an amino acid sequence having more than about any of 80%, 85%, 90%, or 99% identity to the amino acid sequence of SEQ ID NO.1. In some embodiments, the immunosuppressive molecule is (or derived from) a PDL-1 molecule comprising the amino acid sequence of SEQ ID NO:2. In some embodiments, the immunosuppressive molecule is (or derived from) a PDL-1 molecule comprising an amino acid sequence having more than about any of 80%, 85%, 90%, or 99% identity to the amino acid sequence of SEQ ID NO.2.

The lipid bilayer can comprise the immunosuppressive molecules in any suitable amount or concentration that is functionally greater than produced by the producer cell in the absence of introduction of exogenous nucleic acids encoding the immunosuppressive molecules. In some embodiments, the lipid bilayer comprises the immunosuppressive molecules in an amount sufficient to improve delivery and expression of the transgene encoded by an engineered viral vector as compared to the same vector that is not administered in conjunction with an EV engineered to contain the immunosuppressive molecules. As explained in greater detail in connection with the method of producing the EVs, the EVs comprising sufficient concentration of immunosuppressive molecules in the lipid bilayer can be provided by engineering the host (producer) cell to overexpress the immunosuppressive molecules as compared to the native host cell. Thus, in some embodiments, the lipid bilayer of the EVs provided herein comprises one or more (or all) of the immunosuppressive molecules in an amount greater than the same EV produced from the same host cell that has not been engineered to overexpress the immunosuppressive molecules. For instance, the lipid bilayer provided herein comprises one or more (or all) of the immunosuppressive molecules in an amount greater than the same EV produced from the same host cell that has not been engineered to overexpress the immunosuppressive molecules by about 2x or more, by about 3x or more, by about 5x or more, by about 10x or more, by about 20x or more, by about 50x or more, or even about 100x or more (e.g., about 1000x or more). In some embodiments, the host cell is engineered to overexpress one or more (or all) of the immunosuppressive molecules by about 2x or more, about 3x or more, about 5x or more, about 10x or more, about 20x or more, about 50x or more, or even about 100x or more (e.g., about 1000x or more) than the same host cell that is not engineered to overexpress the immunosuppressive molecules. As explained above, in some embodiments, the host cell is a non-tumor host cell engineered to overexpress the immunosuppressive molecules, and the lipid bilayer is a non-tumor EV lipid bilayer, such as a non-tumor exosomal lipid bilayer, from a non-tumor cell engineered to overexpress the immunosuppressive molecules. In a particular embodiments, the lipid bilayer is an EV lipid bilayer (e.g., an exosomal lipid bilayer or an EV) from a 293 cell (e.g., HEK293 or any variation thereof, such as HEK293E, HEK293F, HEK293T, etc.) engineered to overexpress the immunosuppressive molecules. The amount of immunosuppressive molecules on the surface of EVs (e.g., in the lipid bilayer of the EV) can be determined using any of various techniques known in the art. For instance, ELISA can be used to measure the amount of such molecules on the surface of vectors and determine the relative amounts of such molecules on different vectors.

The EVs provided herein can have any suitable particle size. Typically, the EVs will have a size in the range of about 30-600 nm, such as about 50-300 nm, with an average particle size in the range of about 75-150 nm, such as about 80-120 nm (e.g., about 90-115 nm) as measured using a NANOSIGHT™ NS300 (Malvern Instruments, Malvern, United Kingdom) following the manufacturer’s protocol.

### Other Lipid Bilayer Moieties

The EVs provided herein can further include additional moieties in the lipid bilayer as desired to provide different functions. For instance, the lipid bilayer can be engineered to contain membrane surface proteins that target the vector to a desired cell or tissue type, for instance, a molecule that specifically binds to a ligand or receptor on a desired cell type. By engineering the EVs provided herein to contain lipid bilayer-associated targeting moieties (e.g. targeting proteins) that bind to ligands or receptors on a desired cell type, the EV enable more precise targeting to tolerogenic environments; for example, the liver, spleen or thymus. In some embodiments, the lipid bilayer of the EV can be engineered to include a moiety that specifically or preferentially binds a surface protein expressed specifically or preferentially on liver cells (e.g., a protein, such as a membrane-bound antigen binding domain (e.g., domain of clone 8D7, BD Biosciences), that specifically binds asialoglycoprotein receptor 1(ASGR1)). In some embodiments, the targeting molecules is an antibody or antigen-binding fragment thereof, such as scFvs (single-chain variable fragments, composed of a fusion of the variable regions of the heavy and light chains of an immunoglobulin) or Fabs (antigen-binding fragments, composed of one constant and one variable domain from each heavy and light chain of the antibody). In some embodiments, the targeting molecule is a nanobodies: an antibody fragment consisting of a single monomeric variable antibody domain that targets specific proteins or cell types. In some embodiments, the targeting molecule is a protein, a polypeptide or a polysaccharide that specifically bind to desired targets or target cells.

In some embodiments, the targeting molecule targets MHC class I or MHC class II mismatches between donor tissue and a recipient. Such targeting may be used in treating or preventing tissue rejection or graft versus host disease.

As explained in greater detail in connection with the method of producing the EVs, such a lipid bilayer can be provided by engineering host cells (producer cells) to express high levels of a membrane bound targeting moiety. Thus, in some embodiments, the invention provides an EV comprising a lipid bilayer wherein the lipid bilayer comprises an immunosuppressive molecule and a targeting molecule.

The EV can further comprise additional elements that improve effectiveness or efficiency of the EV, or improve production. For example, exogenous expression of Tetraspanin CD9 in producer cells can improve vector production without degrading vector performance (Shiller et al., Mol Ther Methods Clin Dev, (2018) 9:278-287). Thus, the EV might include CD9 in the lipid bilayer. However, in some embodiments, the EV is substantially or completely free of elements that significantly impair the efficiency or effectiveness of the EV for inducing immune tolerance in an individual, render the vector unsuitable for use in humans (e.g., under FDA regulations), or substantially impair EV production.

In some embodiments, the EV is “empty” meaning that the interior of the EV does not contain any molecules heterologous to the cell from which it was made other than the immunosuppressive molecules and targeting molecules as described herein. One skilled in the art would recognize that the EV would contain cellular elements such as cytosol from the producer cell. In some embodiments, the EV is produced from a producer cell which contains no additional heterologous molecules other than the one or more immunosuppressive molecules and targeting molecules. In some embodiments, the EV contains no additional molecules heterologous to the cell from which it was derived other than the one or more immunosuppressive molecules and targeting molecules. In some embodiments, the EVs of the invention do not encapsulate viral vectors (e.g., AAV, HSV, or lentivirus).

### Therapeutic Agents

The invention provides methods for inducing immune tolerance to an agent in an individual wherein an effective amount of an EV, engineered to comprise one or more immunosuppressive molecules in its lipid bilayer, is administered in conjunction with the agent. In some embodiments, the agent is a polypeptide, a nucleic acid, a polypeptide-nucleic acid complex, a viral vector, a liposome, a cell (e.g., a cell therapy agent), or transplanted cells or tissue. In some embodiments, administering the EV of the invention in conjunction with the therapeutic agent to an individual induces immune tolerance of the therapeutic agent in the individual to allow for repeat dosing (i.e., one or more additional administrations following an initial administration.

In some embodiments, the agent is a therapeutic polypeptide. In some embodiments, wherein the therapeutic polypeptide is an enzyme, a hormone, an antibody, an antibody fragment, a clotting factor, a growth factor, a receptor, or a functional derivative thereof. Examples of therapeutic polypeptides include but are not limited to, Factor VIII, Factor IX, myotubularin, survival motor neuron protein (SMN), retinoid isomerohydrolase (RPE65), NADH-ubiquinone oxidoreductase chain 4, Choroideremia protein (CHM), huntingtin, alpha-galactosidase A, acid beta-glucosidase, alpha-glucosidase, ornithine transcarbomylase, argininosuccinate synthetase, β-globin, γ-globin, phenylalanine hydroxylase, and adrenoleukodystrophy protein (ALD).

In some embodiments, the agent is a nucleic acid encoding a therapeutic polypeptide or a therapeutic nucleic acid. Examples of nucleic acids encoding a therapeutic polypeptide include, but not limited to, nucleic acids encoding Factor VIII, Factor IX, myotubularin, survival motor neuron protein (SMN), retinoid isomerohydrolase (RPE65), NADH-ubiquinone oxidoreductase chain 4, Choroideremia protein (CHM), huntingtin, alpha-galactosidase A, acid beta-glucosidase, alpha-glucosidase, ornithine transcarbomylase, argininosuccinate synthetase, β-globin, γ-globin, phenylalanine hydroxylase, and adrenoleukodystrophy protein (ALD). Examples of thereapeutic nucleic acids include, but not limited to, siRNA, miRNA, shRNA, antisense RNA, RNAzyme, and DNAzyme. In some embodiments, the nucleic acid encodes one or more gene editing products; e.g., nucleic acids encoding one or more of CRISPR elements.

In some embodiments, the polypeptide-nucleic acid complex is a gene editing complex; for example, a Cas9 protein associated with the appropriate RNA elements for gene editing.

In some embodiments, the agent is a viral vector; for example, a viral vector for use in gene therapy. In some embodiments, the viral vector is an adeno-associated viral (AAV) vector, a lentiviral vector, an adenoviral vector, a herpes simplex viral vector or a baculovirus vector.

In some embodiments, the viral vector is an AAV vector. AAV is a member of the parvovirus family. Any AAV vector suitable for delivering a transgene can be used in conjunction with the immunosuppressive EV of the invention. The AAV particle can comprise an AAV capsid protein and an AAV viral genome from any serotype. AAV serotypes include, but are not limited to AAV1, AAV2, AAV3, AAV4, AAV5, AAV6, AAV7, AAV8, AAV9, AAV10, AAV11 or AAV12. In some embodiments, the AAV viral particle comprises an AAV viral capsid and an AAV viral genome from the same serotype. In other embodiments, the AAV viral genome and AAV capsid are of different serotypes. For example, the AAV viral capsid may be an AAV6 viral capsid and the AAV viral genome may be an AAV2 viral genome. In some embodiments, the AAV is a self-complementary AAV (scAAV). In some embodiments, the vector is an AAV8 or AAV2/8 vector, particularly scAAV8 or scAAV2/8).

In some embodiments, the viral vector comprises lentiviral particles. Any lentivirus suitable for transgene delivery can be used, including but not limited to human immunodeficiency virus, simian immunodeficiency virus and feline immunodeficiency virus. Typically, the lentiviral vector is non-replicating. The lentiviral vector can be an integrating or non-integrating lentiviral vector. In some embodiments, the lentiviral genome lacks vif, vpr, vpu, tat, rev, nef genes. In some embodiments, the lentiviral genome comprises a heterologous transgene, a 5′ long terminal repeat (LTR) and a 3′ LTR, wherein all or part of a U3 region of the 3′ LTR is removed or replaced by a heterologous regulatory element.

In some embodiments, the EVs are introduced to the individual in conjunction with administering one or more viral capsid proteins or fragments thereof. In some embodiments, the EVs are introduced to the individual in conjunction with administering one or more viral capsid proteins or fragments thereof to induce immune tolerance to the viral vector in the individual. In some embodiments, the one or more viral capsid proteins is an AAV VP1 capsid protein, an AAV VP2 capsid protein, and/or an AAV VP1 capsid protein, or fragment therof. In some embodiments, the one or more viral capsid protein is an adenovirus hexon protein, an adenovirus penton protein, an adenovirus fiber protein, an adenovirus knob protein, or fragment thereof. In some embodiments, the EVs are introduced to the individual in conjunction with administering one or more viral vector envelope proteins or fragments thereof. In some embodiments, the EVs are introduced to the individual in conjunction with administering one or more viral vector envelope proteins or fragments thereof to induce immune tolerance to the viral vector in the individual. In some embodiments, the EVs are introduced to the individual in conjunction with a lentivirus gp120 protein, a lentivirus gp41 protein and/or a protein related to a pseudotyped lentiviral vector. In some embodiments, the EVs are introduced to the individual in conjunction with an HSV gD protein, an HSV gB protein and/or a protein related to a pseudotyped HSV vector.

The viral particle, specifically the viral genome, will include a heterologous nucleic acid (e.g., a transgene) to be delivered (the “payload”) or can be an empty vector. The particular nature of the nucleic acid to be delivered depends on the desired end-use, and the viral vector of the invention is not limited to any particular use or payload. In some embodiments, the payload nucleic acid will express a biological protein, e.g., Factor VIII (e.g., human F8 (UniProtKB - Q2VF45), SQ-FVIII variant of a B-domain-deleted (BDD) human Factor VIII gene (Lind et al., 1995 Eur J Biochem. Aug 15;232(1):19-27)) or other known variants), Factor IX (e.g., human Factor IX UniProtKB - P00740; or human Factor IX (R338L) “Padua” (Monahan et al., 2015 Hum Gene Ther., 26(2): 69-81, or other known variants), myotubularin, SMN, RPE65, NADH-ubiquinone oxidoreductase chain 4, CHM, huntingtin, alpha-galactosidase A, acid beta-glucosidase, alpha-glucosidase, Ornithine transcarbomylase, argininosuccinate synthetase, β-globin, γ-globin, phenylalanine hydroxylase, or ALD. In other embodiments, the payload nucleic acid encodes a reporter molecule, e.g., green fluorescent protein, red fluorescent protein, yellow fluorescent protein, luciferase, alkaline phosphatase, or beta-galactosidase. In still other embodiments, the payload nucleic acid encodes a therapeutic nucleic acid, such as a siRNA, miRNA, shRNA, antisense RNA, RNAzyme, or DNAzyme. In still other embodiments, the payload nucleic acid encodes one or more gene editing gene products, such as an RNA-guided endonuclease (e.g., Cas9, CPF1, etc.), a guide nucleic acid for an RNA-guided endonuclease, a donor nucleic acid, or some combination thereof.

The heterologous nucleic acid can be under control of a suitable promoter, which can be a tissue specific promoter. For example, if the vector is to be delivered to the liver, a liver-specific promoter (e.g., a liver-specific human α1-antitrypsin (hAAT) promoter). Other regulator elements as may be appropriate for a given application also may be included.

In some embodiments, the therapeutic agent is a cell or a tissue. For example, the cell may be a stem cell used in a therapy where engraftment of a stem cell is beneficial. Examples of stem cells include adult stem cells derived from any tissue in the body (e.g., a hematopoietic stem cell, a liver stem cell, a pancreatic stem cell, a muscle stem cell, a cardiomyocyte progenitor cell, a neural stem cell, a mesenchymal stem cell, an adipose stem cell, a bone stem cell, and the like). In some embodiments, the cell is derived from an embryonic stem cell or an induced pluripotent stem cell. In some embodiments, the cell is a pluripotent stem cell or a multipotent stem cell. In some embodiments, the stem cell is an engineered stem cell; for example, the stem cell is engineered to express one or more specific polypeptides.

In some embodiments, the therapeutic agent is a differentiated cell. Examples of differentiated cells include, but not limited to blood cells (e.g., PBMCs), hepatocytes, myocytes, cardiomyocyties, pancreatic cells (e.g., islet cells), ocular cells (e.g., retinal cells and/or corneal cells), inner ear hair cells, neurons, astrocytes, oligodendrocytes, chondrocytes, and bone cells (e.g., osteoblasts). In some embodiments, the differentiated cell is an engineered differentiated cell; for example, the cell is engineered to express one or more specific polypeptides. For example, the cell may be a hepatocyte engineered to express a therapeutic protein such as Factor VIII or Factor IX.

In other embodiments, the cell is obtained from a tissue in a donor for engraftment into a recipient; for example, a hepatocyte, a blood cell, a bone marrow cell, and the like).

In some embodiments, the agent is associated with the EV; for example, the agent associates with the exterior surface of the EV. In some embodiments, the agent is bound to the EV. In some embodiments, the agent is not in the interior of the EV. In some embodiments, the EV and the agent are mixed prior to administration. In some embodiments, the EV and the agent are mixed prior to administration to allow the agent to associate with the EV. In some embodiments, the agent is produced in the same producer cell as the EV and the agent associates with EV in the cell culture supernatant. In some embodiments, the agent is added to the EV producer cell culture supernatant to allow the agent to associate with the EV.

### Compositions

In some aspects, the invention provides compositions comprising the EVs comprising the lipid bilayer-associated immunosuppressive molecules as described herein. In some embodiments, composition comprises the EV and an appropriate carrier, such as a pharmaceutically acceptable carrier such as saline. In some embodiments, the composition comprises the EV and an agent (e.g., a therapeutic agent). In some embodiments, the agent is a polypeptide, a nucleic acid, a polypeptide-nucleic acid complex, a viral vector, a liposome, a cell (e.g., for use in cell therapy) or transplanted cells or tissue. In some embodiments, the composition comprises an agent associated with the EV; for example, the agent associates with the exterior surface of the EV. In some embodiments, the agent is bound to the EV. In some embodiments, the agent is not in the interior of the EV. In some embodiments, the EVs of the invention and the agents of the invention are provided in separate compositions. In some embodiments, the compositions are pharmaceutical compositions. Suitable carriers, formulation buffers, and other excipients for formulation of EVs are known in the art and applicable to the presently provided composition.

Pharmaceutical compositions of the present invention comprise a therapeutically effective amount of EVs formulated in a pharmaceutically acceptable carrier. The phrases “pharmaceutical or pharmacologically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains immunoconjugate and optionally an additional active ingredient will be known to those of skill in the art in light of the present disclosure, as exemplified by Remington’s Pharmaceutical Sciences, 18th Ed. Mack Printing Company, 1990, incorporated herein by reference. Preferred compositions are lyophilized formulations or aqueous solutions. As used herein, “pharmaceutically acceptable carrier” includes any and all solvents, buffers, dispersion media, coatings, surfactants, antioxidants, preservatives (e.g. antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art (see, for example, Remington’s Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

### Applications and Methods of Use

In some aspects, the invention provides methods of treating a disease or disorder in an individual, the method comprising administering an effective amount of an EV to the individual, wherein EV comprises a lipid bilayer comprising one or more immunosuppressive molecules. In some embodiments, the EVs are not administered in conjunction with another agent. In some embodiments, the EVs are administered to generally suppress the individual’s immune system. In some embodiments, the disease or disorder is an autoimmune disease or disorder. In some embodiments, the EV is administered in conjunction with a tissue transplant or cell engraftment. In some embodiments, the EV is not used to treat an autoimmune disease or a Graft versus Host Disease.

In some aspects, the EVs provided herein are useful for inducing immune tolerance for an agent in an individual. In some aspects, the invention provides methods for inducing immune tolerance to an agent in an individual, the method comprising administering an effective amount of an EV to the individual in conjunction with administering the agent to the individual, wherein EV comprises a lipid bilayer comprising one or more immunosuppressive molecules. In some embodiments, the invention provides methods for inducing immune tolerance to an agent in an individual, the method comprising administering a composition comprising an effective amount of an EV and the therapeutic agent to the individual, wherein EV comprises a lipid bilayer comprising one or more immunosuppressive molecules. In some embodiments, the invention provides methods for inducing immune tolerance to an agent in an individual, the method comprising administering a composition comprising an effective amount of an EV, wherein EV comprises a lipid bilayer comprising one or more immunosuppressive molecules, and administering an effective amount of the therapeutic agent to the individual. In some embodiments, the method comprises administering an EV of the invention in conjunctions with a therapeutic agent to the individual in a repeat dosing schedule comprising two or more separate administrations of the EV and the therapeutic agent separated by a suitable time interval (e.g., two or more administrations of a dose of the therapeutic agent separated by at least a day, at least a week, at least two weeks, at least three weeks, at least four weeks or a month, at least two months, at least three months, at least six months, or even at least a year or more).

In some embodiments, the EV, which comprises immunosuppressive molecules in the lipid bilayer, can induce immune tolerance to the agent administered to an individual more effectively or efficiently than administration of the agent to the individual in the absence of administering the EV or in conjunction with an EV that is not engineered to comprise the immunosuppressive molecules. In some embodiments, administering the EVs comprising the immunosuppressive molecules in conjunction with a therapeutic agent enhances the efficacy of the therapeutic agent. For example, administration of the EVs comprising immunosuppressive molecules in their lipid bilayers in conjunction with the therapeutic agent can reduce anti-drug antibody (ADA) responses to the therapeutic agent. In some embodiments of the EVs comprising immunosuppressive molecules in their lipid bilayers are believed to reduce the host immune response to the therapeutic agent, or the impact of the host immune response on transgene delivery and/or expression for nucleic acid-based therapies. In some embodiments, the EVs provided herein allows for repeat dosing of the therapeutic agent and/or dosing of subjects with pre-existing immunity to a given therapeutic agent.

In one aspect of the invention, the method comprises administration of the EV in conjunction with a viral vector to an individual; e.g., for use in a gene therapy. In some embodiments, the individual has been previously exposed to the virus (either by natural exposure to the native virus or by prior administration of the viral vector), or an individual that otherwise has a pre-existing immunity to the virus (e.g., a patient that has pre-existing antibodies to the virus). Thus, the method can comprise administering an EV of the invention in conjunctions with a viral vector to the individual in a repeat dosing schedule comprising two or more separate administrations of the EV and the viral vector separated by a suitable time interval (e.g., two or more administrations of a dose of the viral vector separated by at least a day, at least a week, at least two weeks, at least three weeks, at least four weeks or a month, at least two months, at least three months, at least six months, or even at least a year or more).

In one aspect of the invention, the method comprises administration of the EV in conjunction with a cell to an individual; e.g., for use in a cell therapy. In some embodiments, the cell for use in the cell therapy is an allogeneic cell. In some embodiments, the cell for use in the cell therapy is an autologous cell; for an example, an autologous cell that has been manipulated prior to return donor in a manner that may induce an immune response. In some embodiments, the method can comprise administering an EV of the invention in conjunctions with a cell therapy to the individual in a repeat dosing schedule comprising two or more separate administrations of the EV and the cell therapy separated by a suitable time interval (e.g., two or more administrations of a dose of the viral vector separated by at least a day, at least a week, at least two weeks, at least three weeks, at least four weeks or a month, at least two months, at least three months, at least six months, or even at least a year or more).

Typically, the total amount of the immunosuppressive molecule in a dose of the EVs comprising lipid bilayer-associated immunosuppressive molecules will be less than the dose of the immunosuppressive molecule that would be used when administered as a soluble immunosuppressive agent. Thus, for instance, in CTLA4/Ig might be used as an immunosuppressive agent at a dose of 10 mg/kg. However, in some embodiments, a single dose of EVs will have far less of the immunosuppressive agent (e.g., membrane-bound CTLA4), such as less than about 5 mg/kg, less than about 2 mg/kg, less than about 1 mg/kg, or even less than about 0.5 mg/kg (e.g., less than about 0.1 mg/kg). Accordingly, in some embodiments, the EVs comprising lipid bilayer-associated immunosuppressive molecules provided herein minimizes global immunosuppression that results from administration of soluble immunosuppressive agents (e.g., CTLA4/Ig, abatacept). In some embodiments, global immunosuppression is measured within 2-3 weeks after administration as an increase in circulating total anti-IgG antibodies, or an increase in antigen specific antibodies, or activated CD4+ or CD8+ T Cells that are stimulated by antigens other than those derived from the therapeutic agent administered.

In some embodiments, the EVs are administered to an individual in conjunction with administration of a therapeutic agent. The therapeutic agent can be administered to an individual for any ultimate end purpose. In some embodiments, the therapeutic agent is administered in conjunction with the EV to treat a disease or disorder in an individual. In some embodiments, the disease or disorder is a monogenic disease. In some embodiments, the disease or disorder is a lysosomal storage disease. In some embodiments, the disease or disorder is a glycogen storage disease. In some embodiments, the disease or disorder is a hemoglobin disorder. In some embodiments, the disease or disorder is a musculoskeletal disorder. In some embodiments, the disease or disorder is a CNS disease or disorder. In some embodiments, the disease or disorder is a cardiovascular disorder including heart disease or stroke. In some embodiments, the disease is a cancer. In some embodiments, the disease or disorder is an autoimmune disease. In some embodiments, the disease or disorder is treated by tissue transplantation or cell engraftment (e.g., stem cell engraftment).

More specific illustrative, but non-limiting, examples of diseases include myotobularin myopathy, spinal muscular atrophy, Leber congenital amaurosis, hemophilia A and B, Niemann Pick disease (e.g., Niemann Pick A, Niemann Pick B, Niemann Pick C), choroideremia, Huntington’s disease, Batten disease, Leber hereditary optic neuropathy, ornithine transcarbamylase (OTC) deficiency, glycogen storage diseases, Pompe disease, Wilson disease, citrullinemia Type 1, PKU (phenylketonuria), adrenoleukodystrophy, hemoglobin disorders including sickle cell disease, beta thalassemia, central nervous system disorders, and musculoskeletal disorders.

In some embodiments, the therapeutic agent is a therapeutic polypeptide. Examples of therapeutic polypeptides include, but are not limited to, Factor VIII, Factor IX, myotubularin, SMN, RPE65, NADH-ubiquinone oxidoreductase chain 4, CHM, huntingtin, alpha-galactosidase A, acid beta-glucosidase, alpha-glucosidase, Ornithine transcarbomylase, argininosuccinate synthetase, β-globin, γ-globin, phenylalanine hydroxylase, or ALD.

In some embodiments of the method, the therapeutic agent is a nucleic acid that is administered to a subject that has such a disease or disorder or is at risk of developing the disease or disorder (e.g. carries a mutation for the disease or disorder or has a family history of the disease or disorder). Furthermore, when used to treat a disease or disorder, the nucleic acid expresses a therapeutic polypeptide which treats the disease of the subject. By way of non-limiting example, the nucleic acid might encode one or more of the following: Factor VIII, Factor IX, myotubularin, SMN, RPE65, NADH-ubiquinone oxidoreductase chain 4, CHM, huntingtin, alpha-galactosidase A, acid beta-glucosidase, alpha-glucosidase, Ornithine transcarbomylase, argininosuccinate synthetase, β-globin, γ-globin, phenylalanine hydroxylase, or ALD.

In some embodiments, the agent is a viral vector useful for the delivery and expression of a nucleic acid (transgene) to a cell or individual. Thus, the invention provides a method of delivering a nucleic acid (transgene) to a cell or individual by administering the viral vector in conjunction with administering the EV comprising lipid bilayer-associated immunosuppressive molecules to the cell or individual. In some embodiments, the viral vector is an adeno-associated viral (AAV) vector, a lentiviral vector, an adenoviral vector, a herpes simplex viral vector or a baculovirus.

In some embodiments, the viral vector can deliver the nucleic acid (transgene) to the cell or subject more effectively or efficiently in conjunction with the EV than a viral vector without administering the EV engineered to comprise the immunosuppressive molecules. In some embodiments, the more effective or efficient delivery results in a higher viral genome copy per target cell, and/or higher expression of the transgene product (as applicable) in the cell or subject. For instance, in some embodiments, the combination of the EV and the viral vector provides transgene expression levels 3-weeks following administration to a subject that are increased by about 50% or more (about 75% or more, about 100% or more, about 125% or more, about 150% or more, about 175% or more, or even about 200% or more) as compared to that produced by administration of the viral vector alone under the same conditions (e.g., same transgene, same subject, same dose and route of administration, etc., with the only difference being the EV). In some embodiments, the combination of the EV and the viral vector provides transgene provides transgene expression levels 3-weeks following administration to a subject that are increased by about 20% or more (about 50% or more, about 75% or more, about 100% or more, about 125% or more, about 150% or more, about 175% or more, or even about 200% or more) as compared to that produced by administration of the viral vector alone under the same conditions (e.g., same transgene, same subject, same dose and route of administration, etc., with the only difference being the EV).

The viral vector can be administered in conjunction with administration of the EV comprising lipid bilayer-associated immunosuppressive molecules to deliver a nucleic acid (transgene) to a cell or subject for any ultimate end purpose. In some embodiments, this end purpose might be to express the transgene in a cell in vitro for research purposes, or for the production of a protein or other bio-production process. In other embodiments, the viral vector is used to treat a disease or disorder in an individual. The disease or disorder can be any disease or disorder susceptible to treatment by delivery and (if applicable) expression of a nucleic acid or transgene. In some embodiments, the disease or disorder is a monogenic disease. In some embodiments, the disease or disorder is a lysosomal storage disease. In some embodiments, the disease or disorder is a glycogen storage disease. In some embodiments, the disease or disorder is a hemoglobin disorder. In some embodiments, the disease or disorder is a musculoskeletal disorder. In some embodiments, the disease or disorder is a CNS disease or disorder. In some embodiments, the disease or disorder is a cardiovascular disorder including heart disease or stroke. In some embodiments, the disease is a cancer.

More specific illustrative, but non-limiting, examples of diseases include myotobularin myopathy, spinal muscular atrophy, Leber congenital amaurosis, hemophilia A and B, Niemann Pick disease (e.g., Niemann Pick A, Niemann Pick B, Niemann Pick C), choroideremia, Huntington’s disease, Batten disease, Leber hereditary optic neuropathy, ornithine transcarbamylase (OTC) deficiency, glycogen storage diseases, Pompe disease, Wilson disease, citrullinemia Type 1, PKU (phenylketonuria), adrenoleukodystrophy, hemoglobin disorders including sickle cell disease, beta thalassemia, central nervous system disorders, and musculoskeletal disorders. Thus, in some embodiments of the method, the viral vector is administered in conjunction with the EV comprising lipid bilayer-associated immunosuppressive molecules to an individual that has such a disease or disorder or is at risk of developing the disease or disorder (e.g. carries a mutation for the disease or disorder or has a family history of the disease or disorder). Furthermore, when used to treat a disease or disorder, the viral vector comprises a payload nucleic acid the expression of which treats the disease of the subject. By way of non-limiting example, the nucleic acid might encode one or more of the following: Factor VIII, Factor IX, myotubularin, SMN, RPE65, NADH-ubiquinone oxidoreductase chain 4, CHM, huntingtin, alpha-galactosidase A, acid beta-glucosidase, alpha-glucosidase, Ornithine transcarbomylase, argininosuccinate synthetase, β-globin, γ-globin, phenylalanine hydroxylase, or ALD.

In some embodiments, the therapeutic agent is a therapeutic nucleic acid for the treatment of disease or any other purpose. In some embodiments, the therapeutic nucleic acid is a siRNA, miRNA, shRNA, antisense RNA, RNAzyme, or DNAzyme. In some embodiments, the therapeutic nucleic acid is encoded on a viral vector. In some embodiments, the viral vector is an adeno-associated viral (AAV) vector, a lentiviral vector, an adenoviral vector, a herpes simplex viral vector or a baculovirus.

In some embodiments, the therapeutic agent is a gene editing agent. In some embodiments the therapeutic agent is a nucleic acid or viral vector encoding gene editing elements. In some embodiments, the gene editing agent is an RNA-guided endonuclease (e.g., Cas9 or Cpf1), one or more guide sequences for the RNA-guided endonuclease, and/or one or more donor sequences. In some embodiments, the nucleic acid encoding the gene editing elements are encoded on a viral vector. In some embodiments, the viral vector is an adeno-associated viral (AAV) vector, a lentiviral vector, an adenoviral vector, a herpes simplex viral vector or a baculovirus.

In some embodiments, the therapeutic agent is a cell for use in a cell therapy. In some embodiments, the cell is a stem cell or a differentiated cell. Examples of stem cells include adult stem cells derived from any tissue in the body (e.g., a hematopoietic stem cell, a liver stem cell, a pancreatic stem cell, a muscle stem cell, a cardiomyocyte progenitor cell, a neural stem cell, a mesenchymal stem cell, an adipose stem cell, a bone stem cell, and the like). In some embodiments, the cell is derived from an embryonic stem cell or an induced pluripotent stem cell. In some embodiments, the cell is a pluripotent stem cell or a multipotent stem cell. In some embodiments, the stem cell is an engineered stem cell; for example, the stem cell is engineered to express one or more specific polypeptides.

In some embodiments, the therapeutic agent is a differentiated cell. Examples of differentiated cells include, but not limited to blood cells (e.g., PBMCs), hepatocytes, myocytes, cardiomyocyties, pancreatic cells (e.g., islet cells), ocular cells (e.g., retinal cells and/or corneal cells), neurons, astrocytes, oligodendrocytes, bone cells (e.g., osteoblasts). In some embodiments, the differentiated cell is an engineered differentiated cell; for example, the cell is engineered to express one or more specific polypeptides. For example, the cell may be a hepatocyte engineered to express a therapeutic protein such as Factor VIII or Factor IX.

In some embodiments, the EVs comprising lipid bilayer-associated immunosuppressive molecules are administered in conjunction with a cell-based or tissue-based therapy; for example, a stem-cell therapy or a tissue or organ transplant therapy. In some embodiments, the EVs comprising lipid bilayer-associated immunosuppressive molecules are administered in conjunction with a cell-based or tissue-based therapy to ameliorate or prevent a graft versus host disease (GvHD). In some embodiments, the EVs comprising lipid bilayer-associated immunosuppressive molecules are not used in conjunction with a cell-based or tissue-based therapy to ameliorate or prevent a graft versus host disease (GvHD).

In some embodiments, the EVs comprising lipid bilayer-associated immunosuppressive molecules are administered to an individual with an autoimmune disease. In some embodiments, the EVs comprising lipid bilayer-associated immunosuppressive molecules are administered to an individual with an autoimmune disease in conjunction with another therapeutic agent. In some embodiments, the EVs comprising lipid bilayer-associated immunosuppressive molecules are administered to an individual with an autoimmune disease without administration of another therapeutic agent to treat the autoimmune disease. Examples of autoimmune diseases include, but are not limited to, rheumatoid arthritis, systemic lupus erythematosus, inflammatory bowel disease, multiple sclerosis, type 1 diabetes mellitus, Guillain-Barre syndrome, chronic inflammatory demyelinating polyneuropathy, psoriasis, Grave’s disease, Hashimoto’s thyroiditis, myasthenia gravis, and vasculitis. In some embodiments, the EVs comprising lipid bilayer-associated immunosuppressive molecules are not for use in an individual with an autoimmune disease.

In some embodiments, the EVs comprising lipid bilayer-associated immunosuppressive molecules are administered to an individual where suppression of an immune response is beneficial; for example, but not limited to, individuals suffering from a systemic inflammatory response syndrome such as a cytokine storm syndrome. The systemic inflammatory response can be triggered by an infection (e.g., a viral infection, a bacterial infection, a fungal infection) or by certain drugs such as biologics including antibodies, gene therapies, cell-based therapies (e.g., CAR-T therapies). In some embodiments, the EVs comprising lipid bilayer-associated immunosuppressive molecules are administered to an individual suffering from sepsis.

In any of the foregoing methods, the individual can be any individual, such as a human, a non-human primate, or other mammal including a rodent (e.g., a mouse, a rat, a guinea pig, a hamster), a rabbit, a dog, a cat, a horse, a cow, a pig, a sheep, a frog, or a bird.

In any of the foregoing methods of treatment, a therapeutically effective amount of the EV and/or the agent is administered to the individual by any suitable route of administration. The effective dose and route of administration will depend upon the indication, and can be determined by the practitioner. In some embodiments, the EVs and/or agent is delivered systemically; for example, intravenously, intra-arterially, intraperitoneally, subcutaneously, orally, or by inhalation. In other embodiments, the EVs and/or agent is delivered directly to a tissue (e.g., an organ, a tumor, etc.), or is administered to the CNS (e.g., intrathecally, to the spinal cord, to a specific part of the brain such as a ventricle, the hypothalamus, the pituitary, the cerebrum, the cerebellum, etc.).

In some embodiments of the invention, the EV is administered to the individual before, at the same time, or after administration of the agent. In some embodiments, the EV is administered to the individual at the same time as the agent. In some embodiments, administration of the EV in conjunction with the agent is repeated. In some embodiments, administration of the EV in conjunction with the agent is repeated one time, two times, three times, four times, five times, six times, seven times, eight times, nine times, ten times or more than ten times. In some embodiments, the EVs are administered in conjunction with the agent one time followed by repeat administrations of the agent without administration of the EV.

The EV comprising the lipid bilayer-associated immunosuppressive molecules are used as part of a composition comprising the EV and an appropriate carrier, such as a pharmaceutically acceptable carrier such as saline. In some embodiments, the EV and the agent (e.g., a therapeutic agent) are used together as part of the same composition. In some embodiments, the agent is a polypeptide, a nucleic acid, a polypeptide-nucleic acid complex, a viral vector, a liposome, a cell, or transplanted cells or tissue. In some embodiments, the composition comprises an agent associated with the EV; for example, the agent associates with the exterior surface of the EV. In some embodiments, the agent is bound to the EV. In some embodiments, the agent is in the interior of the EV. In some embodiments, the agent is not in the interior of the EV. In some embodiments, the EV and the agent (e.g., a therapeutic agent) are used together as separate compositions. Suitable carriers, formulation buffers, and other excipients for formulation of EVs are known in the art and applicable to the presently provided composition.

An exemplary treatment is a method for treating hemophilia B comprises administering to an individual in need of treatment an EV provided herein in conjunction with a viral vector comprising a heterologous transgene encoding a human Factor IX (FIX) protein (e.g., human Factor IX UniProtKB - P00740; human Factor IX (R338L) “Padua” (Monahan et al., (2015) Hum Gene Ther., 26(2):69-81, or other known variants), and wherein the EV is an engineered lipid bilayer comprising CTLA-4 and PD-L1. In a more particular embodiment, the viral vector is AAV (e.g., AAV8 or AAV2/8, or scAAV8 or scAAV2/8). In some embodiments, the EV is engineered to contain CTLA-4 and PD-L1 (e.g., an EV from a producer cell (e.g., an HEK293 cell) engineered to overexpress CTLA-4 and PD-L1). In some embodiments, CTLA-4 comprises or is derived from a CTLA comprising the amino acid sequence of SEQ ID NO: 1. In some embodiments, the CTLA-4 comprises an amino acid sequence having more than about any of 80%, 85%, 90%, or 99% identity to the amino acid sequence of SEQ ID NO.1. In some embodiments, CTLA-4 comprises or is derived from a CTLA comprising the amino acid sequence of SEQ ID NO:5. In some embodiments, the CTLA-4 comprises an amino acid sequence having more than about any of 80%, 85%, 90%, or 99% identity to the amino acid sequence of SEQ ID NO.5. In some embodiments, CTLA-4 comprises or is derived from a CTLA comprising the amino acid sequence of SEQ ID NO:7. In some embodiments, the CTLA-4 comprises an amino acid sequence having more than about any of 80%, 85%, 90%, or 99% identity to the amino acid sequence of SEQ ID NO.7. In some embodiments, CTLA-4 comprises or is derived from a CTLA comprising the amino acid sequence of SEQ ID NO:9. In some embodiments, the CTLA-4 comprises an amino acid sequence having more than about any of 80%, 85%, 90%, or 99% identity to the amino acid sequence of SEQ ID NO.9. In some embodiments, the PDL-1 comprises or is derived from a PDL-1 comprising the amino acid sequence of SEQ ID NO:2. In some embodiments, the PDL-1 comprises an amino acid sequence having more than about any of 80%, 85%, 90%, or 99% identity to the amino acid sequence of SEQ ID NO.2. In some embodiments, the PDL-1 comprises or is derived from a PDL-1 comprising the amino acid sequence of SEQ ID NO: 13. In some embodiments, the PDL-1 comprises an amino acid sequence having more than about any of 80%, 85%, 90%, or 99% identity to the amino acid sequence of SEQ ID NO.13. In some embodiments, the EV and/or the viral vector are delivered to the liver, and the heterologous transgene includes a liver-specific promoter. In some embodiments, the EV and the vector is administered intravenously, optionally to the hepatic artery. In some embodiments, the vector will be administered in a dose of 2 × 10¹¹ to 2 × 10¹² vector genomes (vg) per kilogram bodyweight of the subject (e.g., 2 × 10¹¹ to 8 × 10¹¹ or 3 × 10¹¹ to 6 × 10¹¹ vector genomes (vg) per kilogram bodyweight of the subject). In some embodiments, the method comprises administering 2 or more doses of the EV and the viral vector or the viral vector alone (e.g., 3 or more doses, 4 or more doses, or 5 or more doses) with an interval of at least one day (at least a day, at least a week, at least two weeks, at least three weeks, at least four weeks or a month, at least two months, at least three months, at least six months, or even at least a year or more) between the doses.

In another particular embodiment, a method of treating hemophilia A is provided, which method comprises administering to a subject in need of treatment the EV and a viral vector comprising a heterologous transgene encoding a human Factor VIII (e.g., human F8 (UniProtKB - Q2VF45), SQ-FVIII variant of a B-domain-deleted (BDD) human F8 gene (Lind et al., (1995) Eur J Biochem. Aug 15;232(1):19-27), or other known variant). In some embodiments, the EV comprises an engineered lipid bilayer comprising CTLA-4 and PD-L1. In a more particular embodiment, the viral vector is AAV (e.g., AAV8 or scAAV8, or scAAV8 or scAAV2/8). In some embodiments, the EV is produced from a host cell (e.g., an HEK293 cell) engineered to overexpress CTLA-4 and PD-L1. In some embodiments, CTLA-4 comprises or is derived from a CTLA comprising the amino acid sequence of SEQ ID NO: 1. In some embodiments, the CTLA-4 comprises an amino acid sequence having more than about any of 80%, 85%, 90%, or 99% identity to the amino acid sequence of SEQ ID NO: 1. In some embodiments, CTLA-4 comprises or is derived from a CTLA comprising the amino acid sequence of SEQ ID NO:5. In some embodiments, the CTLA-4 comprises an amino acid sequence having more than about any of 80%, 85%, 90%, or 99% identity to the amino acid sequence of SEQ ID NO:5. In some embodiments, CTLA-4 comprises or is derived from a CTLA comprising the amino acid sequence of SEQ ID NO:7. In some embodiments, the CTLA-4 comprises an amino acid sequence having more than about any of 80%, 85%, 90%, or 99% identity to the amino acid sequence of SEQ ID NO:7. In some embodiments, CTLA-4 comprises or is derived from a CTLA comprising the amino acid sequence of SEQ ID NO:9. In some embodiments, the CTLA-4 comprises an amino acid sequence having more than about any of 80%, 85%, 90%, or 99% identity to the amino acid sequence of SEQ ID NO:9. In some embodiments, the PDL-1 comprises or is derived from a PDL-1 comprising the amino acid sequence of SEQ ID NO:2. In some embodiments, the PDL-1 comprises an amino acid sequence having more than about any of 80%, 85%, 90%, or 99% identity to the amino acid sequence of SEQ ID NO.2. In some embodiments, the PDL-1 comprises or is derived from a PDL-1 comprising the amino acid sequence of SEQ ID NO:13. In some embodiments, the PDL-1 comprises an amino acid sequence having more than about any of 80%, 85%, 90%, or 99% identity to the amino acid sequence of SEQ ID NO:13. In some embodiments, the EV and/or the viral vector is delivered to the liver, and the heterologous transgene includes a liver-specific promoter. In some embodiments, the EV and/or the vector is administered intravenously, optionally to the hepatic artery. In some embodiments, the vector will be administered in a dose of 2 × 10¹¹ to 2 × 10¹² vector genomes (vg) per kilogram bodyweight of the subject (e.g., 2 × 10¹¹ to 8 × 10¹¹ or 3 × 10¹¹ to 6 × 10¹¹ vector genomes (vg) per kilogram bodyweight of the subject). In some embodiments, the method comprises administering 2 or more doses of the EV and the viral vector or the viral vector alone (e.g., 3 or more doses, 4 or more doses, or 5 or more doses) with an interval of at least one day (at least a day, at least a week, at least two weeks, at least three weeks, at least four weeks or a month, at least two months, at least three months, at least six months, or even at least a year or more) between the doses.

### Manufacturing

The EVs provided herein can be produced by any suitable method. Non-limiting example are provided by US 9829483B2 and US 2013/0202559, incorporated herein by reference.

One particularly advantageous method involves producing the EVs from a producer cell line that has been engineered to overexpress the immunosuppressive molecules desired to be included in the lipid bilayer of the EV. Thus, provided herein is a method of preparing an EV with a lipid bilayer comprising immunosuppressive molecules, as described herein, by (a) culturing producer cells under conditions to generate EVs, wherein the producer cells comprise a nucleic acid encoding one or more one or more membrane-bound immunosuppressive molecules, and (b) collecting the EVs.

### Producer Cell Engineering

Any producer cell suitable for the conventional production of the EVs can be used to produce the EVs of the invention. In some embodiments, the producer cells are mammalian cells. In some embodiments, the producer cells are human cells. Suitable producer cells include, but are not limited to, 293 cells (e.g., HEK293, HEK293E, HEK293F, HEK293T, and the like), Hela cells, and Per.C6. The producer cells can be engineered to express the desired immunosuppressive molecules by any suitable method. In some embodiments of the invention, immunosuppressive molecules are expressed by transfection, either stably or transiently, of an exogenous nucleic acid (e.g., plasmids or other vectors) encoding the immunosuppressive molecules into producer cells. By expression of such exogenous nucleic acids, the producer cells overexpress the immunosuppressive molecules as compared to the same producer cell that has not been transfected with exogenous nucleic acids encoding the immunosuppressive molecules, and an EV that buds from the producer cell, in turn, has increased amounts of the immunosuppressive molecules as compared to an EV budding from the same producer cell that has not been engineered to overexpress the immunosuppressive molecules. In some embodiments, the host cell that is engineered to overexpress the immunosuppressive molecules by about 2x or more, about 3x or more, about 5x or more, about 10x or more, about 20x or more, about 50x or more, or even about 100x or more than the same host cell that is not engineered to overexpress the immunosuppressive molecules.

Expression of the immunosuppressive molecules can be driven by a promoter, such as a constitutive promoter (e.g., a CMV promoter). In some embodiments, the gene encoding the effector molecule is followed by polyadenylation signal (e.g., a hemoglobin polyadenylation signal) downstream of the effector molecule coding region. In some embodiments, an intron is inserted downstream of the promoter. For example, a hemoglobin derived artificial intron downstream of the promoter may be employed to increase effector molecule production. The method for transient transfections includes but is not limited to calcium phosphate transfection. The method to produce stable cell lines expressing single or combined immune modulators includes but is not limited to retroviral gene transfer or concatemer transfection followed by selection (Throm et al. (2009) Blood, 113(21): 5104-5110). The producer cells are engineered in this way to express individual immunosuppressive molecules, or to express different combinations of immunosuppressive molecules, as may be desired in the EV. The producer cells also can be engineered in other ways known in the art to increase productivity. For example, the producer cells can be engineered to overexpress Tetraspanin CD9 to improve vector production (Shiller et al., (2018) Mol Ther Methods Clin Dev, 9:278-287).

### Production of EVs Comprising Lipid Bilayer-Associated Immunosuppressive Molecules

The EVs described herein can be produced from the engineered producer cells by any suitable technique. For example, the media from the producer cells is collected and EVs are purified. In some embodiments, EVs of 50-200 nm in diameter are preferentially isolated from media from the producer cells for use, e.g., by chromatography purification methods such as size exclusion chromatography, affinity chromatography, or ion exchange chromatography. In some embodiments, EVs of about 25 to about 500 nm in diameter are isolated. In some embodiments, EVs of between about 15 to about 50 nm, about 50 to about 75 nm, about 75 to about 100 nm, about 100 to about 150 nm, about 150 to about 200 nm, about 200 to about 250 nm, about 250 to about 300 nm, about 300 to about 350 nm, about 350 to about 400 nm, about 400 to about 450 nm, or about 450 to about 500 nm in diameter. In some embodiments, the EVs in the media can be clarified or filtered using depth filtration and or combining 0.44 or 0.2 µM sterile filters, to remove cells and cellular debris and colloidal particles. Alternatively, media from producer cells can be clarified using tangential flow filtration to remove residual impurities.

When the EVs includes a targeting moiety as described herein, the targeting moiety can be used as an affinity ligand to aid in isolation/purification. Likewise, the immunosuppressive molecules may be used as an affinity ligand to aid in isolation/purification.

EVs are harvested after an empirically determined length of time, and then purified using any of various techniques known in the art. Purifications techniques can include but are not limited to ion-exchange chromatography, size exclusion chromatography, affinity chromatography, and tangential flow filtration. Ultracentrifugation, including continuous ultracentrifugation, may be used to purify the EVs.

The amounts of EVs produced per liter of producer cells can be increased using various methods. These methods can include but are not limited to adding molecules that suppress apoptosis, or suspend cell division to the producer cell during fermentation. Molecules or compounds that alter the lipid composition of producer cell membranes may also be used to increase EV production per liter. Additionally, compounds or molecules that increase EV production, including membrane fusigenic molecules.

Thus, in some embodiments, the invention provides a method of producing an EV as described herein, the method comprising (a) culturing producer cells under conditions to generate EVs, wherein the producer cells comprise nucleic acids encoding one or more one or more membrane bound immunosuppressive molecules, and (b) collecting the EVs. The EVs can have any of the features and elements described herein with respect to the EVs of the invention. Furthermore, the producer cells can have any of the features and elements described in the previous sections, and the method of producing the EVs can further include steps of providing the producer cells by, for instance, transforming the producer cells with nucleic acids encoding the one or more membrane-bound immunosuppressive molecules. In some embodiments, the host cell is engineered to overexpress the immunosuppressive molecules (e.g., comprises one or more exogenous nucleic acids encoding the immunosuppressive molecules) by about 2x or more, about 3x or more, about 5x or more, about 10x or more, about 20x or more, about 50x or more, or even about 100x or more than the same host cell that is not engineered to overexpress the immunosuppressive molecules. In some embodiments, the host cell is a non-tumor cell, such as a 293 cell (e.g., HEK293, HEK293T, HEK293E, HEK293F, etc.) or Per.C6.

Collection of the EVs can comprise isolating the EVs from the culture fluid of the cultured viral producer cells. In some embodiments, EVs are collected by separation of the EVs from the cell culture by ultracentrifugation or other suitable method. The method preferably avoids the use of detergents. Furthermore, the method preferably minimizes or avoids lysis of the producer cells prior to collection of the EV, as the lysis of the producer cells will release host cell proteins and nucleic acid into the culture.

### Kits

The present invention also provides kits for administering EVs comprising lipid bilayer-associated immunosuppressive molecules in conjunction with administration of an agent (e.g., a therapeutic agent) described herein to a cell or subject according to the methods of the invention. The kits may comprise any EV of the invention.

In some embodiments, the kits further include instructions for EV. The kits described herein may further include other materials desirable from a commercial and user standpoint, including other buffers, diluents, filters, needles, syringes, and package inserts with instructions for performing any methods described herein. Suitable packaging materials may also be included and may be any packaging materials known in the art, including, for example, vials (such as sealed vials), vessels, ampules, bottles, jars, flexible packaging (e.g., sealed Mylar or plastic bags), and the like. These articles of manufacture may further be sterilized and/or sealed. In some embodiments, the kits comprise instructions for treating a disease disorder described herein using any of the methods and/or EVs described herein. The kits may include a pharmaceutically acceptable carrier suitable for injection into the individual, and one or more of: a buffer, a diluent, a filter, a needle, a syringe, and a package insert with instructions for performing injections into the mammal.

In some embodiments, the kits further contain one or more of the buffers and/or pharmaceutically acceptable excipients described herein (e.g., as described in REMINGTON’S PHARMACEUTICAL SCIENCES (Mack Pub. Co., N.J. 1991). In some embodiments, the kits include one or more pharmaceutically acceptable excipients, carriers, solutions, and/or additional ingredients described herein. The kits described herein can be packaged in single unit dosages or in multidosage forms. The contents of the kits are generally formulated as sterile and can be lyophilized or provided as a substantially isotonic solution.

### EXEMPLARY EMBODIMENTS

Embodiment 1. A method of inducing immune tolerance to an agent in an individual, the method comprising administering an effective amount of an EV to the individual in conjunction with administering the agent to the individual, wherein EV comprises a lipid bilayer comprising one or more immunosuppressive molecules.

Embodiment 2. The method of embodiment 2, wherein the one or more immunosuppressive molecules comprise one or more immune checkpoint proteins.

Embodiment 3. The method of embodiment 1 or 2, wherein the one or more immunosuppressive molecules comprise one or more of CTLA4, B7-1, B7-2, PD-1, PD-L1, PD-L2, CD28, VISTA, TIM-3, GAL9, TIGIT, CD155, LAG3, VISTA, BTLA or HVEM.

Embodiment 4. The method of embodiment 1 or 2, wherein the one or more immunosuppressive molecules targets CD40 or CD40L.

Embodiment 5. The method of embodiment 4, wherein the immunosuppressive molecule is an antibody that binds CD40 or CD40L.

Embodiment 6. The method of any one of embodiments 1-5, wherein the lipid bilayer comprises two or more, three or more, or four or more different immunosuppressive molecules; or comprises two or more, three or more, or four or more different checkpoint proteins.

Embodiment 7. The method of any one of embodiments 1-6, wherein the lipid bilayer comprises CTLA4 and PD-L1; CTLA and PD-L2; CTLA-4 and VISTA; PD-L1 and PD-L2; PD-L1 and VISTA; PD-L2 and VISTA; CTLA4 and PD-L1 and PD-L2; CTLA4 and PD-L1 and VISTA; CTLA4 and PD-L2 and VISTA; PD-L1 and PD-L2 and VISTA; or CTLA4 and PD-L1 and PD-L1 and VISTA.

Embodiment 8. The method of any one of embodiments 1-7, wherein one or more of the immunosuppressive molecules comprises a transmembrane domain.

Embodiment 9. The method of embodiment 8, wherein the transmembrane domain is a PDGFR transmembrane domain, an EGFR transmembrane domain or a murine CTLA4 transmembrane domain.

Embodiment 10. The method of any one of embodiments 1-9, wherein the lipid bilayer further comprises a targeting molecule.

Embodiment 11. The method of embodiment 10, wherein the targeting molecule confers cell- or tissue-specificity to the EV.

Embodiment 12. The method of embodiment 10 or 11, wherein the targeting molecule confers specificity of the method to the liver, spleen, and/or thymus.

Embodiment 13. The method of embodiment 10 or 11, wherein the targeting molecule targets MHC class I or MHC class II mismatches between donor tissue and the individual.

Embodiment 14. The method of any one of embodiments 10-13, wherein the targeting molecule is an antibody.

Embodiment 15. The method of any one of embodiments 10-14, wherein the one or more targeting molecules comprises a transmembrane domain.

Embodiment 16. The method of any one of embodiments 1-15, wherein the EV is produced from a producer cell engineered to express the one or more immunosuppressive molecules.

Embodiment 17. The method of any one of embodiments 1-16, wherein the EV is produced from a producer cell engineered to express the one or more immunosuppressive molecules.

Embodiment 18. The method of any one of embodiments 1-17, wherein the EV is produced from a producer cell engineered to express one or more of CTLA4, B7-1, B7-2, PD-1, PD-L1, PD-L2, CD28, VISTA, TIM-3, GAL9, TIGIT, CD155, LAG3, VISTA, BTLA, HVEM, an anti-CD40 antibody or an anti-CD40L antibody.

Embodiment 19. The method of embodiment 18, wherein the producer cell is a human embryonic kidney 293 (HEK 293) cell, HeLa cell, or a Per.C6 cell.

Embodiment 20. The method of any one of embodiments 1-19, wherein the EV is produced from a producer cell which contains no additional heterologous molecules other than the one or more immunosuppressive molecules and targeting molecules.

Embodiment 21. The method of any one of embodiments 1-20, wherein the EV contains no additional molecules heterologous to the cell from which it was derived other than the one or more immunosuppressive molecules and targeting molecules.

Embodiment 22. The method of any one of embodiments 1-21, wherein the EV is administered to the individual before, at the same time, or after administration of the agent.

Embodiment 23. The method of any one of embodiments 1-22, wherein the EV is administered to the individual at the same time as administration of the agent.

Embodiment 24. The method of any one of embodiments 1-23, wherein the EV and the agent are in different formulations.

Embodiment 25. The method of any one of embodiments 1-24, wherein the EV and the agent are in the same formulation.

Embodiment 26. The method of embodiment 25, wherein the agent associates with the EV.

Embodiment 27. The method of embodiment 25 or 26, wherein the agent associates with the exterior surface of the EV.

Embodiment 28. The method of any one of embodiments 25-27, wherein the stimulation of immune tolerance facilitates repeat administration of the agent to the individual.

Embodiment 29. The method of embodiment 28, wherein the repeat administration comprises more than about 2 administrations, 3 administrations, 4 administrations, 5 administrations, 6 administrations, 7 administrations, 8 administrations, 9 administrations, or 10 administrations of the agent.

Embodiment 30. The method of any one of embodiments 1-29, wherein the agent is a therapeutic agent.

Embodiment 31. The method of any one of embodiments 1-30, wherein the agent is a polypeptide, a nucleic acid, a polypeptide-nucleic acid complex, a viral vector, a liposome, a cell, or transplanted cells or tissue.

Embodiment 32. The method of embodiment 31, wherein the agent is a therapeutic polypeptide.

Embodiment 33. The method of embodiment 32, wherein the therapeutic polypeptide is an enzyme, a hormone, an antibody, an antibody fragment, a clotting factor, a growth factor, a receptor, or a functional derivative thereof.

Embodiment 34. The method of embodiment 32 or 33, wherein the therapeutic polypeptide is Factor VIII, Factor IX, myotubularin, survival motor neuron protein (SMN), retinoid isomerohydrolase (RPE65), NADH-ubiquinone oxidoreductase chain 4, Choroideremia protein (CHM), huntingtin, alpha-galactosidase A, acid beta-glucosidase, alpha-glucosidase, ornithine transcarbomylase, argininosuccinate synthetase, β-globin, γ-globin, phenylalanine hydroxylase, adrenoleukodystrophy protein (ALD), dystrophin, a truncated dystrophin, Niemann Pick C protein (NPC-1), an anti-VEGF agent, or a functional variant thereof.

Embodiment 35. The method of embodiment 31, wherein the agent is a nucleic acid encoding a therapeutic polypeptide or a therapeutic nucleic acid.

Embodiment 36. The method of embodiment 35, wherein the nucleic acid encodes Factor VIII, Factor IX, myotubularin, survival motor neuron protein (SMN), retinoid isomerohydrolase (RPE65), NADH-ubiquinone oxidoreductase chain 4, Choroideremia protein (CHM), huntingtin, alpha-galactosidase A, acid beta-glucosidase, alpha-glucosidase, ornithine transcarbomylase, argininosuccinate synthetase, β-globin, γ-globin, phenylalanine hydroxylase, or adrenoleukodystrophy protein (ALD).

Embodiment 37. The method of embodiment 35, wherein the therapeutic nucleic acid is a siRNA, miRNA, shRNA, antisense RNA, RNAzyme, or DNAzyme.

Embodiment 38. The method of embodiment 37, wherein the nucleic acid encodes one or more gene editing products.

Embodiment 39. The method of embodiment 31, wherein the polypeptide-nucleic acid complex is a gene editing complex.

Embodiment 40. The method of embodiment 31, wherein the agent is a viral vector or a capsid protein thereof.

Embodiment 41. The method of embodiment 40, wherein the viral vector is an adeno-associated viral (AAV) vector, a lentiviral vector, an adenoviral vector, a herpes simplex viral vector or a baculovirus vector.

Embodiment 42. The method of embodiment 31, wherein the agent is a cell used in cell therapy.

Embodiment 43. The method of embodiment 42, wherein the cell is a stem cell, an induced pluripotent cell (iPS), or a differentiated cell.

Embodiment 44. The method of embodiment 42 or 43, wherein the cell is a pluripotent cell or a multipotent cell.

Embodiment 45. The method of embodiment 43 or 44, wherein the cell is an embryonic stem cell or an adult stem cell.

Embodiment 46. The method of embodiment 45, wherein the cell is a hematopoietic stem cell, a liver stem cell, a muscle stem cell, a cardiomyocyte stem cell, a neural stem cell, a bone stem cell, a mesenchymal stem cell, or an adipose stem cell.

Embodiment 47. The method of embodiment 42 or 43, wherein the cell is a blood cell, a hepatocyte, a myocyte, a cardiomyocyte, a pancreatic cell, an islet cell, an ocular cell, a neural cell, an astrocyte, an oligodendrocyte, an inner ear hair cell, a chondrocyte, or an osteoblast.

Embodiment 48. The method of any one of embodiments 42-47, wherein the cell is allogeneic to the individual.

Embodiment 49. The method of any one embodiments 1-48, wherein the individual is a human.

Embodiment 50. A method of treating a disease or disorder in an individual, the method comprising administering an effective amount of an EV to the individual, wherein EV comprises a lipid bilayer comprising one or more immunosuppressive molecules.

Embodiment 51. The method of embodiment 50, wherein the disease or disorder is an autoimmune disease or disorder.

Embodiment 52. The method of embodiment 50, wherein the EV is administered in conjunction with a tissue transplant or cell engraftment.

Embodiment 53. A method of treating a disease or disorder in an individual, the method comprising administering an effective amount of an EV to the individual in conjunction with administering an agent to the individual, wherein EV comprises a lipid bilayer comprising one or more immunosuppressive molecules, and wherein the agent treats the disease or disorder.

Embodiment 54. The method of any one of embodiments 50-53, wherein the one or more immunosuppressive molecules comprise one or more immune checkpoint proteins.

Embodiment 55. The method of any one of embodiments 50-54, wherein the one or more immunosuppressive molecules comprise one or more of CTLA4, B7-1, B7-2, PD-1, PD-L1, PD-L2, CD28, VISTA, TIM-3, GAL9, TIGIT, CD155, LAG3, VISTA, BTLA or HVEM.

Embodiment 56. The method of any one of embodiments 50-54, wherein the one or more immunosuppressive molecules targets CD40 or CD40L.

Embodiment 57. The method of embodiment 56, wherein the immunosuppressive molecule is an antibody that binds CD40 or CD40L.

Embodiment 58. The method of any one of embodiments 50-57, wherein the lipid bilayer comprises two or more, three or more, or four or more different immunosuppressive molecules; or comprises two or more, three or more, or four or more different checkpoint proteins.

Embodiment 59. The method of any one of embodiments 50-58, wherein the lipid bilayer comprises CTLA4 and PD-L1; CTLA and PD-L2; CTLA-4 and VISTA; PD-L1 and PD-L2; PD-L1 and VISTA; PD-L2 and VISTA; CTLA4 and PD-L1 and PD-L2; CTLA4 and PD-L1 and VISTA; CTLA4 and PD-L2 and VISTA; PD-L1 and PD-L2 and VISTA; or CTLA4 and PD-L1 and PD-L1 and VISTA.

Embodiment 60. The method of any one of embodiments 50-59, wherein one or more of the immunosuppressive molecules comprises a transmembrane domain.

Embodiment 61. The method of embodiment 60, wherein the transmembrane domain is a PDGFR transmembrane domain, an EGFR transmembrane domain or a murine CTLA4 transmembrane domain.

Embodiment 62. The method of any one of embodiments 50-61, wherein the lipid bilayer further comprises a targeting molecule.

Embodiment 63. The method of embodiment 62, wherein the targeting molecule confers cell- or tissue-specificity to the EV.

Embodiment 64. The method of embodiment 62 or 63, wherein the targeting molecule confers specificity of the method to the liver, spleen, and/or thymus.

Embodiment 65. The method of embodiment 62 or 63, wherein the targeting molecule targets MHC class I or MHC class II mismatches between donor tissue and the individual.

Embodiment 66. The method of any one of embodiments 62-65, wherein the targeting molecule is an antibody.

Embodiment 67. The method of any one of embodiments 62-66, wherein the one or more targeting molecules comprises a transmembrane domain.

Embodiment 68. The method of any one of embodiments 50-67, wherein the EV is produced from a producer cell engineered to express the one or more immunosuppressive molecules.

Embodiment 69. The method of any one of embodiments 50-68, wherein the EV is produced from a producer cell engineered to express the one or more immunosuppressive molecules.

Embodiment 70. The method of any one of embodiments 50-69, wherein the EV is produced from a producer cell engineered to express one or more of CTLA4, B7-1, B7-2, PD-1, PD-L1, PD-L2, CD28, VISTA, TIM-3, GAL9, TIGIT, CD155, LAG3, VISTA, BTLA, HVEM, an anti-CD40 antibody or an anti-CD40L antibody.

Embodiment 71. The method of any one of embodiments 50-70, wherein the producer cell is a human embryonic kidney 293 (HEK 293) cell, HeLa cell, or a Per.C6 cell.

Embodiment 72. The method of any one of embodiments 50-71, wherein the EV is produced from a producer cell which contains no additional heterologous molecules other than the one or more immunosuppressive molecules and targeting molecules.

Embodiment 73. The method of any one of embodiments 50-72, wherein the EV contains no additional molecules heterologous to the cell from which it was derived other than the one or more immunosuppressive molecules and targeting molecules.

Embodiment 74. The method of embodiment 92 or 93, wherein the EV is administered to the individual before, at the same time, or after administration of the agent.

Embodiment 75. The method of any one of embodiments 52-74, wherein the EV is administered to the individual at the same time as administration of the agent.

Embodiment 76. The method of any one of embodiments 52-75, wherein the EV and the agent are in different formulations.

Embodiment 77. The method of any one of embodiments 52-75, wherein the EV and the agent are in the same formulation.

Embodiment 78. The method of embodiment 77, wherein the agent associates with the EV.

Embodiment 79. The method of embodiment 77 or 78, wherein the agent associates with the exterior surface of the EV.

Embodiment 80. The method of any one of embodiments 52-79, wherein the stimulation of immune tolerance facilitates repeat administration of the agent to the individual.

Embodiment 81. The method of embodiment 80, wherein the repeat administration comprises more than about 2 administrations, 3 administrations, 4 administrations, 5 administrations, 6 administrations, 7 administrations, 8 administrations, 9 administrations, or 10 administrations of the agent.

Embodiment 82. The method of any one of embodiments 52-81, wherein the agent is a therapeutic agent.

Embodiment 83. The method of any one of embodiments 52-82, wherein the agent is a polypeptide, a nucleic acid, a polypeptide-nucleic acid complex, a viral vector, a liposome, or transplanted cells or tissue.

Embodiment 84. The method of embodiment 83, wherein the agent is a therapeutic polypeptide.

Embodiment 85. The method of embodiment 84, wherein the therapeutic polypeptide is an enzyme, a hormone, an antibody, an antibody fragment, a clotting factor, a growth factor, a receptor, or a functional derivative thereof.

Embodiment 86. The method of embodiment 84 or 85, wherein the therapeutic polypeptide is Factor VIII, Factor IX, myotubularin, survival motor neuron protein (SMN), retinoid isomerohydrolase (RPE65), NADH-ubiquinone oxidoreductase chain 4, Choroideremia protein (CHM), huntingtin, alpha-galactosidase A, acid beta-glucosidase, alpha-glucosidase, ornithine transcarbomylase, argininosuccinate synthetase, γ-globin, β-globin, phenylalanine hydroxylase, or adrenoleukodystrophy protein (ALD).

Embodiment 87. The method of embodiment 83, wherein the agent is a nucleic acid encoding a therapeutic polypeptide or a therapeutic nucleic acid.

Embodiment 88. The method of embodiment 87, wherein the nucleic acid encodes Factor VIII, Factor IX, myotubularin, survival motor neuron protein (SMN), retinoid isomerohydrolase (RPE65), NADH-ubiquinone oxidoreductase chain 4, Choroideremia protein (CHM), huntingtin, alpha-galactosidase A, acid beta-glucosidase, alpha-glucosidase, ornithine transcarbomylase, argininosuccinate synthetase, γ-globin, β-globin, phenylalanine hydroxylase, or adrenoleukodystrophy protein (ALD).

Embodiment 89. The method of embodiment 88, wherein the therapeutic nucleic acid is a siRNA, miRNA, shRNA, antisense RNA, RNAzyme, or DNAzyme.

Embodiment 90. The method of embodiment 83, wherein the nucleic acid encodes one or more gene editing products.

Embodiment 91. The method of embodiment 90, wherein the polypeptide-nucleic acid complex is a gene editing complex.

Embodiment 92. The method of embodiment 83, wherein the agent is a viral vector or a capsid protein thereof.

Embodiment 93. The method of embodiment 92, wherein the viral vector is an adeno-associated viral (AAV) vector, a lentiviral vector, an adenoviral vector, a herpes simplex viral vector or a baculovirus.

Embodiment 94. The method of embodiment 83, wherein the agent is a cell used in cell therapy.

Embodiment 95. The method of embodiment 94, wherein the cell is a stem cell, an induced pluripotent cell (iPS), or a differentiated cell.

Embodiment 96. The method of embodiment 94 or 95, wherein the cell is a pluripotent cell or a multipotent cell.

Embodiment 97. The method of embodiment 95 or 96, wherein the cell is an embryonic stem cell or an adult stem cell.

Embodiment 98. The method of embodiment 97, wherein the cell is a hematopoietic stem cell, a liver stem cell, a muscle stem cell, a cardiomyocyte stem cell, a neural stem cell, a bone stem cell, a mesenchymal stem cell, or an adipose stem cell.

Embodiment 99. The method of embodiment 94 or 95, wherein the cell is a blood cell, a hepatocyte, a myocyte, a cardiomyocyte, a pancreatic cell, an islet cell, an ocular cell, a neural cell, an astrocyte, an oligodendrocyte, an inner ear hair cell, a chondrocyte, or an osteoblast.

Embodiment 100. The method of any one of embodiments 94-99, wherein the cell is allogeneic to the individual.

Embodiment 101. The method of any one embodiments 50-100, wherein the individual is a human.

Embodiment 102. A composition comprising an extracellular vesicle (EV) and one or more pharmaceutically acceptable excipients for inducing immune tolerance to an agent in an individual, wherein EV comprises a lipid bilayer comprising one or more immunosuppressive molecules, and a therapeutic agent.

Embodiment 103. The composition of embodiment 102, wherein the agent is a polypeptide, a nucleic acid, a polypeptide-nucleic acid complex, a viral vector, a liposome, a cell, or transplanted cells or tissue.

Embodiment 104. The composition of embodiment 102 or 103, wherein the agent associates with the EV.

Embodiment 105. The composition of any one of embodiments 102-104, wherein the agent associates with the exterior surface of the EV.

Embodiment 106. The composition of any one of embodiments 102-105, wherein the one or more immunosuppressive molecules comprise one or more immune checkpoint proteins.

Embodiment 107. The composition of any one of embodiments 102-106, wherein the one or more immunosuppressive molecules comprise one or more of CTLA4, B7-1, B7-2, PD-1, PD-L1, PD-L2, CD28, VISTA, TIM-3, GAL9, TIGIT, CD155, LAG3, VISTA, BTLA, or HVEM.

Embodiment 108. The composition of any one of embodiments 102-107, wherein the one or more immunosuppressive molecules targets CD40 or CD40L.

Embodiment 109. The composition of embodiment 108, wherein the immunosuppressive molecule is an antibody that binds CD40 or CD40L.

Embodiment 110. The composition of any one of embodiments 102-109, wherein the lipid bilayer comprises two or more, three or more, or four or more different immunosuppressive molecules; or comprises two or more, three or more, or four or more different checkpoint proteins.

Embodiment 111. The composition of any one of embodiments 102-110, wherein the lipid bilayer comprises CTLA4 and PD-L1; CTLA and PD-L2; CTLA-4 and VISTA; PD-L1 and PD-L2; PD-L1 and VISTA; PD-L2 and VISTA; CTLA4 and PD-L1 and PD-L2; CTLA4 and PD-L1 and VISTA; CTLA4 and PD-L2 and VISTA; PD-L1 and PD-L2 and VISTA; or CTLA4 and PD-L1 and PD-L1 and VISTA.

Embodiment 112. The composition of any one of embodiments 102-111, wherein one or more of the immunosuppressive molecules comprises a transmembrane domain.

Embodiment 113. The composition of embodiment 112, wherein the transmembrane domain is a PDGFR transmembrane domain, an EGFR transmembrane domain or a murine CTLA4 transmembrane domain.

Embodiment 114. The composition of any one of embodiments 102-113, wherein the lipid bilayer further comprises a targeting molecule.

Embodiment 115. The composition of embodiment 114, wherein the targeting molecule confers cell- or tissue-specificity to the EV.

Embodiment 116. The composition of embodiment 114 or 115, wherein the targeting molecule confers specificity of the EV to the liver, spleen, and/or thymus.

Embodiment 117. The composition of any one of embodiments 114-116, wherein the targeting molecule targets MHC class I or MHC class II mismatches between donor tissue and the individual.

Embodiment 118. The composition of any one of embodiments 114-117, wherein the targeting molecule is an antibody.

Embodiment 119. The composition of any one of embodiments 114-118, wherein the one or more targeting molecules comprises a transmembrane domain.

Embodiment 120. The composition of any one of embodiments 102-119, wherein the EV is produced from a producer cell engineered to express the one or more immunosuppressive molecules.

Embodiment 121. The composition of any one of embodiments 102-120, wherein the EV is produced from a producer cell engineered to express the one or more immunosuppressive molecules.

Embodiment 122. The composition of any one of embodiments 102-121, wherein the EV is produced from a producer cell engineered to express one or more of CTLA4, B7-1, B7-2, PD-1, PD-L1, PD-L2, CD28, VISTA, TIM-3, GAL9, TIGIT, CD155, LAG3, VISTA, BTLA. HVEM, an anti-CD40 antibody or an anti-CD40L antibody.

Embodiment 123. The composition of any one of embodiments 102-122, wherein the producer cell is a human embryonic kidney 293 (HEK 293) cell, HeLa cell, or a Per.C6 cell.

Embodiment 124. The composition of any one of embodiments 102-123, wherein the EV is produced from a producer cell which contains no additional heterologous molecules other than the one or more immunosuppressive molecules and targeting molecules.

Embodiment 125. The composition of any one of embodiments 102-124, wherein the EV contains no additional molecules heterologous to the cell from which it was derived other than the one or more immunosuppressive molecules and targeting molecules.

Embodiment 126. A method of producing the composition of any of embodiments 102-125, the method comprising a) culturing EV producer cells in vitro under conditions to generate EVs, wherein the EV producer cells comprise nucleic acids encoding one or more one or more membrane-bound immunosuppressive molecules, b) collecting the EVs, and c) formulating the EVs with the agent.

Embodiment 127. The method of embodiment 126, wherein the EV producer cells comprise exogenous nucleic acids encoding the membrane-bound immunosuppressive molecules.

Embodiment 128. The method of embodiment 126 or 127, wherein the membrane-bound immunosuppressive molecules comprise one or more of CTLA4, B7-1, B7-2, PD-1, PD-L1, PD-L2, CD28, VISTA, TIM-3, GAL9, TIGIT, CD155, LAG3, VISTA, BTLA or HVEM.

Embodiment 129. The method of embodiment 126 or 127, wherein the one or more immunosuppressive molecules targets CD40 or CD40L.

Embodiment 130. The method of embodiment 129, wherein the immunosuppressive molecule is an antibody that binds CD40 or CD40L.

Embodiment 131. The method of any one of embodiments 126-130, wherein one or more of the immunosuppressive molecules comprises a transmembrane domain.

Embodiment 132. The method of embodiment 131, wherein the transmembrane domain is a PDGFR transmembrane domain, an EGFR transmembrane domain or a murine CTLA4 transmembrane domain.

Embodiment 133. The method of any one of embodiments 126-132, wherein the lipid bilayer further comprises a targeting molecule.

Embodiment 134. The method of embodiment 133, wherein the targeting molecule confers cell- or tissue-specificity to the EV.

Embodiment 135. The method of embodiment 133 or 134, wherein the targeting molecule confers specificity of the EV to the liver, spleen, and/or thymus.

Embodiment 136. The method of embodiment 133 or 134, wherein the targeting molecule targets MHC class I or MHC class II mismatches between donor tissue and the individual.

Embodiment 137. The method of any one of embodiments 133-136, wherein the targeting molecule is an antibody.

Embodiment 138. The method of any one of embodiments 133-137, wherein the one or more targeting molecules comprises a transmembrane domain.

Embodiment 139. The method of any one of embodiments 126-138, wherein the EV is produced from a producer cell engineered to express the one or more immunosuppressive molecules.

Embodiment 140. The method of any one of embodiments 126-139, wherein the EV is produced from a producer cell engineered to express one or more of CTLA4, B7-1, B7-2, PD-1, PD-L1, PD-L2, CD28, VISTA, TIM-3, GAL9, TIGIT, CD155, LAG3, VISTA, BTLA or HVEM.

Embodiment 141. The method of embodiment 140, wherein the producer cell is a human embryonic kidney 293 (HEK 293) cell, HeLa cell, or a Per.C6 cell.

Embodiment 142. The method of any one of embodiments 126-141, wherein the EV is produced from a producer cell which contains no additional heterologous molecules other than the one or more immunosuppressive molecules and targeting molecules.

Embodiment 143. The method of any one of embodiments 126-142, wherein the EV contains no additional molecules heterologous to the cell from which it was derived other than the one or more immunosuppressive molecules and targeting molecules.

Embodiment 144. A producer cell for producing an immunosuppressive EV, wherein EV comprises a lipid bilayer comprising one or more immunosuppressive molecules, wherein the one or more immunosuppressive molecules are membrane-bound.

Embodiment 145. The producer cell of embodiment 144, wherein the producer cell is engineered to express the one or more immunosuppressive molecules.

Embodiment 146. The producer cell of embodiment 144 or 145, wherein the producer cell is engineered to express one or more of CTLA4, B7-1, B7-2, PD-1, PD-L1, PD-L2, CD28, VISTA, TIM-3, GAL9, TIGIT, CD155, LAG3, VISTA, BTLA or HVEM.

Embodiment 147. The producer cell of embodiment 144 or 145, wherein the one or more immunosuppressive molecules targets CD40 or CD40L.

Embodiment 148. The producer cell of embodiment 147, wherein the immunosuppressive molecule is an antibody that binds CD40 or CD40L.

Embodiment 149. The producer cell of any one of embodiments 144-148, wherein one or more of the immunosuppressive molecules comprises a transmembrane domain.

Embodiment 150. The producer cell of embodiment 149, wherein the transmembrane domain is a PDGFR transmembrane domain, an EGFR transmembrane domain or a murine CTLA4 transmembrane domain.

Embodiment 151. The producer cell of any one of embodiments 144-150, wherein the lipid bilayer further comprises a targeting molecule.

Embodiment 152. The producer cell of embodiment 151, wherein the targeting molecule confers cell- or tissue-specificity to the EV.

Embodiment 153. The producer cell of embodiment 151 or 152, wherein the targeting molecule confers specificity of the EV to the liver, spleen, and/or thymus.

Embodiment 154. The producer cell of embodiment 152 or 153, wherein the targeting molecule targets MHC class I or MHC class II mismatches between donor tissue and the individual.

Embodiment 155. The producer cell of any one of embodiments 151-154, wherein the targeting molecule is an antibody.

Embodiment 156. The producer cell of any one of embodiments 151-155, wherein the one or more targeting molecules comprises a transmembrane domain.

Embodiment 157. The producer cell of any one of embodiments 151-156, wherein the cell comprises nucleic acid encoding the one or more immunosuppressive molecule and/or the one or more targeting molecule.

Embodiment 158. The producer cell of embodiment 157, wherein the nucleic acid encoding the one or more immunosuppressive molecule and/or the one or more targeting molecule is stably integrated into the genome of the cell.

Embodiment 159. The producer cell of any one of embodiments 144-158, wherein the producer cell is a mammalian cell.

Embodiment 160. The producer cell of any one of embodiments 144-159, wherein the producer cell is a human cell.

Embodiment 161. The producer cell of any one of embodiments 144-160, wherein the producer cell is a human embryonic kidney 293 (HEK 293) cell, HeLa cell, or a Per.C6 cell.

Embodiment 162. The producer cell of any one of embodiments 144-161, wherein the producer cell contains no additional heterologous molecules other than the one or more immunosuppressive molecules and targeting molecules.

Embodiment 163. An extracellular vesicle (EV) for inducing immune tolerance to an agent in an individual, wherein EV comprises a lipid bilayer comprising one or more immunosuppressive molecules.

Embodiment 164. The EV of embodiment 163, wherein the one or more immunosuppressive molecules comprise one or more immune checkpoint proteins.

Embodiment 165. The EV of embodiment 163 or 164, wherein the one or more immunosuppressive molecules comprise one or more of CTLA4, B7-1, B7-2, PD-1, PD-L1, PD-L2, CD28, VISTA, TIM-3, GAL9, TIGIT, CD155, LAG3, VISTA, BTLA, or HVEM.

Embodiment 166. The EV of embodiment 163 or 164, wherein the one or more immunosuppressive molecules targets CD40 or CD40L.

Embodiment 167. The EV of embodiment 166, wherein the immunosuppressive molecule is an antibody that binds CD40 or CD40L.

Embodiment 168. The EV of any one of embodiments 162-167, wherein the lipid bilayer comprises two or more, three or more, or four or more different immunosuppressive molecules; or comprises two or more, three or more, or four or more different checkpoint proteins.

Embodiment 169. The EV of any one of embodiments 163-165, wherein the lipid bilayer comprises CTLA4 and PD-L1; CTLA and PD-L2; CTLA-4 and VISTA; PD-L1 and PD-L2; PD-L1 and VISTA; PD-L2 and VISTA; CTLA4 and PD-L1 and PD-L2; CTLA4 and PD-L1 and VISTA; CTLA4 and PD-L2 and VISTA; PD-L1 and PD-L2 and VISTA; or CTLA4 and PD-L1 and PD-L1 and VISTA.

Embodiment 170. The EV of any one of embodiments 163-169, wherein one or more of the immunosuppressive molecules comprises a transmembrane domain.

Embodiment 171. The EV of embodiment 170, wherein the transmembrane domain is a PDGFR transmembrane domain, an EGFR transmembrane domain, or a murine CTLA4 transmembrane domain.

Embodiment 172. The EV of any one of embodiments 163-171, wherein the lipid bilayer further comprises a targeting molecule.

Embodiment 173. The EV of embodiment 172, wherein the targeting molecule confers cell- or tissue-specificity to the EV.

Embodiment 174. The EV of embodiment 172 or 173, wherein the targeting molecule confers specificity of the EV to the liver, spleen, and/or thymus.

Embodiment 175. The EV of embodiment 172 or 173, wherein the targeting molecule targets MHC class I or MHC class II mismatches between donor tissue and the individual.

Embodiment 176. The EV of any one of embodiments 172-175, wherein the targeting molecule is an antibody.

Embodiment 177. The EV of any one of embodiments 172-176, wherein the one or more targeting molecules comprises a transmembrane domain.

Embodiment 178. The EV of any one of embodiments 163-177, wherein the EV is produced from a producer cell engineered to express the one or more immunosuppressive molecules.

Embodiment 179. The EV of any one of embodiments 163-178, wherein the EV is produced from a producer cell engineered to express the one or more immunosuppressive molecules.

Embodiment 180. The EV of any one of embodiments 163-179, wherein the EV is produced from a producer cell engineered to express one or more of CTLA4, B7-1, B7-2, PD-1, PD-L1, PD-L2, CD28, VISTA, TIM-3, GAL9, TIGIT, CD155, LAG3, VISTA, BTLA. HVEM, an anti-CD40 antibody or an anti-CD40L antibody.

Embodiment 181. The EV of any one of embodiments 163-180, wherein the producer cell is a human embryonic kidney 293 (HEK 293) cell, HeLa cell, or a Per.C6 cell.

Embodiment 182. The EV of any one of embodiments 163-181, wherein the EV is produced from a producer cell which contains no additional heterologous molecules other than the one or more immunosuppressive molecules and targeting molecules.

Embodiment 183. The EV of any one of embodiments 163-182, wherein the EV contains no additional molecules heterologous to the cell from which it was derived other than the one or more immunosuppressive molecules and targeting molecules.

Embodiment 184. A composition comprising the EV of any one of embodiments 163-183 and one or more pharmaceutically acceptable excipients.

### EXAMPLES

### Example 1: EV Production

EVs engineered to express lipid bilayer-associated CTLA4 and PD-L1 are produced using producer cells. Producer HEK293T cells are co-transfected with pCMV.mCTLA-4 and pCMV.mPDL-1 expression vectors. pCMV.mCTLA-4 contains the murine CTLA-4 cDNA sequence driven by a CMV promoter (Sino Biological catalog # MG50503-UT). pCMV.mPDL-1 contains the murine PDL-1 cDNA sequence driven by a CMV promoter (Sino Biological catalog # MG50010-M).

EVs are shed into the culture media along with a portion of the cell membrane (lipid bilayer), and are collected from culture media. Producer cell cultures are centrifuged, and producer cells are separated from the supernatant. EVs are isolated and purified from the supernatant using 2-step ultracentrifugation, and resuspended in PBS, result in a population of EVs with an average particle size of about 100 nm.

The levels of murine CTLA-4 and PDL-1 on EVs are quantified using bead based FACS analysis using fluorescent-labelled antibodies: anti-murine CTLA-4 (anti-CTLA-4 PECy7, Abcam catalog number ab134090) and anti-murine PDL-1 (anti-PDL-1- PE-A, Abcam catalog number ab213480).

### Example 2: In Vivo Gene Transfer in Mice

The following example illustrates the use of the vectors produced in Example 1 for gene transfer in vivo in C57B⅙ Mice.

C57B⅙ Mice (fourteen male and fourteen female) are injected intravenously with 1 × 10¹⁰ vector genomes and 1-200 µg/kg EVs engineered to express CTLA4 and PD-L1 (Exo-mISM). Dosing groups included: 1) PBS only (vehicle control), 2) AAV8-hFIX, 3) AAV8- hFIX + Exo-mISM, and 4) Exo-mISM.

At week three post-dosing, mice are bled and analyzed for (a) human FIX levels (VisuLize™ Factor IX (FIX) Antigen Kit, Affinity Biologicals), (b) AAV8-binding antibodies (BAb) by ELISA using anti-AAV8 IgG, and (c) AAV8- neutralizing antibodies (NAb) using a neutralizing antibody assay (Meliani et al. (2015) Hum Gene Ther Methods, 26:45-53) . The in vitro neutralizing assay is used to measure the titer of antibodies that prevent test AAV vectors from infecting target cells. Briefly, the assay entails incubating an optimized multiplicity of infection (MOI) of test vector containing a reporter gene such as Luciferase, with serial dilutions of test antibodies, then allowing the vector to infect a permissive target cell. The amount of fluorescence from infected cells is measured after 24 hours and indicates the titer of neutralizing antibodies. The neutralizing titer of the sample is determined as the first dilution at which 50% or greater inhibition of the luciferase expression is measured.

At week three post-dosing, two male and two female mice from each group are sacrificed and livers from animals are analyzed for vector genome copy number (VGCN) per cell by qPCR. Tissue DNA is extracted from whole organ using the Magna Pure 96 DNA and viral DNA small volume kit (Roche Diagnostics, Indianapolis IN) according to the manufacturer’s instructions. Vector genome copy number is quantified by TaqMan real-time PCR with the ABI PRISM 7900 HT sequence detector (Thermo Fisher Scientific, Waltham, MA). The mouse RPP30 gene is used as normalizer.

The remaining animals are then (three weeks post-dosing) administered 1 × 10¹⁰ vg of the same AAV vector that is initially administered for each dose group. At week six, mice are again bled and analyzed for human FIX levels, AAV8-binding antibodies (BAb), and AAV8-neutralizing antibodies (NAb) by the same protocols. All remaining animals are then sacrificed and livers from animals are analyzed for vector genomes per cell by qPCR using the prior protocol. Reduced immune responses to AAV and human FIX indicative of induction of immune tolerance to AAV and to human FIX.

An increase in blood level of Factor IX (FIX) compared to control animals is indicative of successful gene transfer and expression. Increased expression of FIX following a second delivery of AAV in the AAV + empty Exo-ISM animals is indicative of induction of immune tolerance and/or enhanced efficacy due to the combination of AAV and an EV comprising lipid bilayer-associated immunosuppressive molecules.

Challenge experiments. Dosing groups are identical as above with the addition of these dosing groups 5) 1 × 10⁹ VG AAV8-human Factor IX (AAV8-FIX) vector + 1-200 µg/mL of empty Exo-mISM + 1-400 µg/dose/mouse administered intraperitoneally anti murine PDL-1 antibody such as CD274 (PD-L1, B7-H1) Monoclonal Antibody (mIH5), Functional Grade, eBioscience™, ThermoFisher Scientific Cat. # 241.00, 6) anti PDL-1 antibody alone, 7) 1 × 10⁹ VG AAV8-human Factor IX vector + 1-200 µg/mL of empty Exo-mISM empty Exo-hISM + 1-400 µg/dose/mouse administered intraperitoneally anti-murine CTLA-4 antibody such as CD152 (CTLA-4) Monoclonal Antibody (14D3), Functional Grade, eBioscience™, 8) anti CTLA-4 antibody alone. Analysis is identical as above.

Adoptive transfer example experiments will determine whether transgene FIX tolerance induced by Exo-mISMs in mice is mediated by CD4+T cells, or different immune cells found in in splenocytes. Experiments are designed similarly to those described in Mingozzi et al., J Clin Invest. 2003, 111(9):1347-56.

Briefly, C56B⅙ Mice (14 male and 14 female) are injected intravenously with 1-200 µg/kg EVs engineered to express murine CTLA4 and PD-L1 (mISM). Dosing groups included: 1) PBS only (vehicle control), 2) AAV8 FIX, 3) AAV8 FIX + empty Exo-mISM, 4) AAV-8 FIX + empty Exo (without mISM).

Splenocytes are harvested from mice in all test groups and purified, then 5 × 10⁷ splenocytes from each animal harvested and purified then injected intravenously into corresponding recipient naive C57b/6 mice. Recipient mice are challenged with subcutaneous injection of hFIX. Two weeks post challenge blood is drawn from recipient mice and analyzed by ELISA for anti-FIX antibodies. Tolerance induction is shown when mice in recipient group 3, receiving AAV + empty Exo-mISM, results in anti-FIX antibody levels that are significantly lower than those produced by recipients of splenocytes from dose groups 2 and 4.

To determine which immune cells are causing tolerance, the same adaptive transfer experiment described above is repeated again except the adoptive transfer is done using CD4+, or other immune cell type that is suspected of inducing tolerance, depleted splenocytes. If the depleted cell type causes tolerance, dose group 3 would no longer show significantly lower anti-FIX antibody levels, then groups 2 and 4.

### Example 3. EV + Biologic Therapy

The following example illustrates the use of the vectors produced in Example 1 for inducing tolerance to a therapeutic agent.

C57B⅙ Mice (14 male and 14 female) are injected intravenously with 1-200 µg/kg EVs engineered to express murine CTLA4 and PD-L1 (mISM). Dosing groups included: 1) PBS only (vehicle control), 2) hFIX, 3) hFIX + empty Exo-mISM, 4) hFIX + empty Exo (without mISM).

Mice are bled weekly and analyzed for indicators of tolerance including: the presence or absence or anti-hFIX antibody responses; levels of IFNγ, IL-2, IL-10, IL10A or IL10B); and T cell and B cell profiles (CD4+. CD8+, Tim 3+, FoxP3+, Tregs).

Challenge experiments. C57B⅙ Mice (14 male and 14 female) are injected intravenously with 1-200 µg/kg EVs engineered to express murine CTLA4 and PD-L1 (mISM). Dosing groups included: 1) PBS only (vehicle control), 2) hFIX, 3) hFIX + empty Exo-mISM, 4) hFIX + empty Exo (without mISM), 5) hFIX + empty Exo-mISM + 1-400 µg/dose/mouse administered intraperitoneally anti murine PDL-1 antibody such as CD274 (PD-L1, B7-H1) Monoclonal Antibody (mIH5), Functional Grade, eBioscience™, ThermoFisher Scientific Cat. # 241.00, 6) anti PDL-1 antibody alone, 7) hFIX vector + empty Exo-hISM + 1-400 µg/dose/mouse administered intraperitoneally anti-murine CTLA-4 antibody such as CD152 (CTLA-4) Monoclonal Antibody (14D3), Functional Grade, eBioscience™, 8) anti CTLA-4 antibody alone. Analysis is identical as in initial experiment.

Adoptive transfer example experiments will determine whether hFIX tolerance induced by Exo-mISMs in mice is mediated by CD4+T cells, or different immune cells found in in splenocytes. Experiments are designed similarly to those described in Mingozzi et al., J Clin Invest. 2003, 111(9):1347-56.

Briefly C57B⅙ Mice (14 male and 14 female) are injected intravenously with 1-200 µg/kg EVs engineered to express murine CTLA4 and PD-L1 (mISM). Dosing groups included: 1) PBS only (vehicle control), 2) hFIX, 3) hFIX + empty Exo-mISM, 4) hFIX + empty Exo (without mISM).

Splenocytes are harvested from mice in all test groups, then 5 X10⁷ are purified and then injected intravenously into corresponding recipient naive C57b/6 mice. Recipient mice are challenged with subcutaneous injection of hFIX. Two weeks post challenge blood is drawn from recipient mice and analyzed by ELISA for anti-FIX antibodies. Tolerance induction is shown when mice in recipient group 3, receiving AAV + empty Exo-mISM, results in anti FIX antibody levels that are significantly lower than those produced by recipients of splenocytes from dose groups 2 and 4.

To determine which immune cells are causing tolerance, the same adaptive transfer experiment described above is repeated again except the adoptive transfer is done using CD4+, or other immune cell type that is suspected of inducing tolerance, depleted splenocytes. If the depleted cell type causes tolerance, dose group 3 would no longer show significantly lower anti-FIX antibody levels, then groups 2 and 4.

### Example 4. EV + AAV-OVA

The following example illustrates the use of AAV8 capsid-Ovalbumin (AAV8 Ova) vectors produced as in Example 1 except using an Ovalbumin transgene for gene transfer to skeletal muscle in vivo in C57B⅙ Mice. A description of the Ova transgene plasmid is found in Wang et al., Blood. 2005, 105(11):4226-34. This experiment shows that empty Exo-mISM co-administered with an AAV8 Ova vector injected into the gastrocnemius muscles of C57B/6 at 1 × 10¹¹ or 1 × 10¹² VG/dose, results in tolerance to the ovalbumin transgene. Tolerance is seen when Ova serum protein, VGCN in the injection site, and mRNA in muscle cells near the injection site are significantly higher in animals that receive the vector + empty Exo-mISM than those that receive vector alone.

Serum Ova quantitation, anti-Ova IgG antibody quantitation, VCGN and Ova mRNA per cell, and quantitation of Ova specific T cells found in blood is analyzed as described in Adriouch et al., Frontiers in Microbiology, 2011, 2(199).

An increase in blood level of Ovalbumin (Ova), or Ova mRNA levels compared to control animals is indicative of successful gene transfer and expression. Increased expression of Ova following a second delivery of AAV in the AAV + empty Exo-mISM animals is indicative of induction of immune tolerance and/or enhanced efficacy due to the combination of AAV and an EV comprising lipid bilayer-associated immunosuppressive molecules.

### Example 5. EV + Cell Therapy

The following illustrates the use of empty Exo-mISM dosed at the time of transplantation of allogeneic islet cells in mice in a Type 1 Diabetes model.

Eleven Male BALB/C mice are used for the in vitro and transplantation studies. Dosing groups are: 1) 600 allogeneic islets transplanted + 1-200 µg/mL of empty Exo-mISM, 2) 600 allogeneic islets transplanted islets with 1-200 µg/mL of empty Exo (no mISM), 3) islets only, 4) PBS. Islet viability and function are measured to assess successful tolerance generation to allogeneic islets. All methods are described in Yamane, et al. Sci Rep 10, 12086 (2020).

Briefly:
C57B/6 mice are donors for islets and BalbC mice recipients and only allogeneic transplants will be performed.
Islets will not be pre-treated instead dosing groups are described above.
Reduced graft inflammation of animals receiving islets transplants with empty Exo-AAV mISM, that is statistically less than graft inflammation in animals that received islets with empty Exo (no mISMs) indicates that empty Exo-mISM reduces inflammation in islet cell intraportal allogeneic transplants generates transplant tolerance leading to better islet transfer engraftment and function.
Significantly sustained reduced blood glucose levels in animals treated with islets + empty Exo-mISM but not with animals that received islets + empty Exo (no mISMs) indicates that Empty Exo-mISM dosed at the time of transplant surgery results in better control of blood glucose levels in this Diabetes Type 1 mouse model.
Significantly longer survival rates in mice in animals treated with islets + empty Exo-mISM but not with animals that received islets + empty Exo (no mISMs) indicates that Empty Exo-mISM dosed at the time of transplant surgery results in longer graft survival.
A single intraperitoneal injection of streptozotocin at a dose of 150 mg/kg body weight is administered to C57/B6 mice to induce diabetes mellitus.

Inflammation will be measured as follows: the inflammatory IFNγ protein levels are measured in tissue samples from liver; characterization of hepatic lymphoid tissues by FACS analysis, T cell infiltrates in liver using immunohistochemistry staining with hematoxylin-eosin. In vivo function of islets is measured using intraperitoneal glucose tolerance test.

Embodiments are described herein, including the best mode of operation. Variations of those embodiments may become apparent to those of ordinary skill in the art upon reading the foregoing description, and such variations are contemplated by applicant. Accordingly, disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

### SEQUENCES

Human CTLA-4: NCBI Reference Sequence: NP_005205.2

MACLGFQRHKAQLNLATRTWPCTLLFFLLFIPVFCKAMHVAQPAVVLASS
RGIASFVCEYASPGKATEVRVTVLRQADSQVTEVCAATYMMGNELTFLDD
SICTGTSSGNQVNLTIQGLRAMDTGLYICKVELMYPPPYYLGIGNGTQIY
VIDPEPCPDSDFLLWILAAVSSGLFFYSFLLTAVSLSKMLKKRSPLTTGV
YVKMPPTEPECEKQFQPYFIPIN(SEQ ID NO: 1)

Human PD-L1: NCBI Reference Sequence: NP_054862.1

MRIFAVFIFMTYWHLLNAFTVTVPKDLYVVEYGSNMTIECKFPVEKQLDL
AALIVYWEMEDKNIIQFVHGEEDLKVQHSSYRQRARLLKDQLSLGNAALQ
ITDVKLQDAGVYRCMISYGGADYKRITVKVNAPYNKINQRILVVDPVTSE
HELTCQAEGYPKAEVIWTSSDHQVLSGKTTTTNSKREEKLFNVTSTLRIN
TTTNEIFYCTFRRLDPEENHTAELVIPELPLAHPPNERTHLVILGAILLC
LGVALTFIFRLRKGRMMDVKKCGIQDTNSKKQSDTHLEET(SEQ ID NO
:2)

Human CTLA-4 nucleic acid

ATGGCTTGCCTTGGATTTCAGCGGCACAAGGCTCAGCTGAACCTGGCTAC
CAGGACCTGGCCCTGCACTCTCCTGTTTTTTCTTCTCTTCATCCCTGTCT
TCTGCAAAGCAATGCACGTGGCCCAGCCTGCTGTGGTACTGGCCAGCAGC
CGAGGCATCGCCAGCTTTGTGTGTGAGTATGCATCTCCAGGCAAAGCCAC
TGAGGTCCGGGTGACAGTGCTTCGGCAGGCTGACAGCCAGGTGACTGAAG
TCTGTGCGGCAACCTACATGATGGGGAATGAGTTGACCTTCCTAGATGAT
TCCATCTGCACGGGCACCTCCAGTGGAAATCAAGTGAACCTCACTATCCA
AGGACTGAGGGCCATGGACACGGGACTCTACATCTGCAAGGTGGAGCTCA
TGTACCCACCGCCATACTACCTGGGCATAGGCAACGGAACCCAGATTTAT
GTAATTGATCCAGAACCGTGCCCAGATTCTGACTTCCTCCTCTGGATCCT
TGCAGCAGTTAGTTCGGGGTTGTTTTTTTATAGCTTTCTCCTCACAGCTG
TTTCTTTGAGCAAAATGCTAAAGAAAAGAAGCCCTCTTACAACAGGGGTC
TATGTGAAAATGCCCCCAACAGAGCCAGAATGTGAAAAGCAATTTCAGCC
TTATTTTATTCCCATCAAT(SEQ ID NO:3)

Human PD-L1 nucleic acid

ATGAGGATATTTGCTGTCTTTATATTCATGACCTACTGGCATTTGCTGAA
CGCATTTACTGTCACGGTTCCCAAGGACCTATATGTGGTAGAGTATGGTA
GCAATATGACAATTGAATGCAAATTCCCAGTAGAAAAACAATTAGACCTG
GCTGCACTAATTGTCTATTGGGAAATGGAGGATAAGAACATTATTCAATT
TGTGCATGGAGAGGAAGACCTGAAGGTTCAGCATAGTAGCTACAGACAGA
GGGCCCGGCTGTTGAAGGACCAGCTCTCCCTGGGAAATGCTGCACTTCAG
ATCACAGATGTGAAATTGCAGGATGCAGGGGTGTACCGCTGCATGATCAG
CTATGGTGGTGCCGACTACAAGCGAATTACTGTGAAAGTCAATGCCCCAT
ACAACAAAATCAACCAAAGAATTTTGGTTGTGGATCCAGTCACCTCTGAA
CATGAACTGACATGTCAGGCTGAGGGCTACCCCAAGGCCGAAGTCATCTG
GACAAGCAGTGACCATCAAGTCCTGAGTGGTAAGACCACCACCACCAATT
CCAAGAGAGAGGAGAAGCTTTTCAATGTGACCAGCACACTGAGAATCAAC
ACAACAACTAATGAGATTTTCTACTGCACTTTTAGGAGATTAGATCCTGA
GGAAAACCATACAGCTGAATTGGTCATCCCAGAACTACCTCTGGCACATC
CTCCAAATGAAAGGACTCACTTGGTAATTCTGGGAGCCATCTTATTATGC
CTTGGTGTAGCACTGACATTCATCTTCCGTTTAAGAAAAGGGAGAATGAT
GGATGTGAAAAAATGTGGCATCCAAGATACAAACTCAAAGAAGCAAAGTG
ATACACATTTGGAGGAGACGTAA(SEQ ID NO:4)

hCTLA-4 with a Y201A point Mutation

MACLGFQRHKAQLNLATRTWPCTLLFFLLFIPVFCKAMHVAQPAVVLASS
RGIASFVCEYASPGKATEVRVTVLRQADSQVTEVCAATYMMGNELTFLDD
SICTGTSSGNQVNLTIQGLRAMDTGLYICKVELMYPPPYYLGIGNGTQIY
VIDPEPCPDSDFLLWILAAVSSGLFFYSFLLTAVSLSKMLKKRSPLTTGV
AVKMPPTEPECEKQFQPYFIPIN(SEQ ID NO:5)

ATGGCTTGCCTTGGATTTCAGCGGCACAAGGCTCAGCTGAACCTGGCTAC
CAGGACCTGGCCCTGCACTCTCCTGTTTTTTCTTCTCTTCATCCCTGTCT
TCTGCAAAGCAATGCACGTGGCCCAGCCTGCTGTGGTACTGGCCAGCAGC
CGAGGCATCGCCAGCTTTGTGTGTGAGTATGCATCTCCAGGCAAAGCCAC
TGAGGTCCGGGTGACAGTGCTTCGGCAGGCTGACAGCCAGGTGACTGAAG
TCTGTGCGGCAACCTACATGATGGGGAATGAGTTGACCTTCCTAGATGAT
TCCATCTGCACGGGCACCTCCAGTGGAAATCAAGTGAACCTCACTATCCA
AGGACTGAGGGCCATGGACACGGGACTCTACATCTGCAAGGTGGAGCTCA
TGTACCCACCGCCATACTACCTGGGCATAGGCAACGGAACCCAGATTTAT
GTAATTGATCCAGAACCGTGCCCAGATTCTGACTTCCTCCTCTGGATCCT
TGCAGCAGTTAGTTCGGGGTTGTTTTTTTATAGCTTTCTCCTCACAGCTG
TTTCTTTGAGCAAAATGCTAAAGAAAAGAAGCCCTCTTACAACAGGGGTC
GCTGTGAAAATGCCCCCAACAGAGCCAGAATGTGAAAAGCAATTTCAGCC
TTATTTTATTCCCATCAAT(SEQ ID NO:6)

hCTLA-4 with no C Terminus

MACLGFQRHKAQLNLATRTWPCTLLFFLLFIPVFCKAMHVAQPAVVLASS
RGIASFVCEYASPGKATEVRVTVLRQADSQVTEVCAATYMMGNELTFLDD
SICTGTSSGNQVNLTIQGLRAMDTGLYICKVELMYPPPYYLGIGNGTQIY
VIDPEPCPDSDFLLWILAAVSSGLFFYSFLLTAVSLSKMLKKRSPLTTGV
(SEQ ID NO:7)

ATGGCTTGCCTTGGATTTCAGCGGCACAAGGCTCAGCTGAACCTGGCTAC
CAGGACCTGGCCCTGCACTCTCCTGTTTTTTCTTCTCTTCATCCCTGTCT
TCTGCAAAGCAATGCACGTGGCCCAGCCTGCTGTGGTACTGGCCAGCAGC
CGAGGCATCGCCAGCTTTGTGTGTGAGTATGCATCTCCAGGCAAAGCCAC
TGAGGTCCGGGTGACAGTGCTTCGGCAGGCTGACAGCCAGGTGACTGAAG
TCTGTGCGGCAACCTACATGATGGGGAATGAGTTGACCTTCCTAGATGAT
TCCATCTGCACGGGCACCTCCAGTGGAAATCAAGTGAACCTCACTATCCA
AGGACTGAGGGCCATGGACACGGGACTCTACATCTGCAAGGTGGAGCTCA
TGTACCCACCGCCATACTACCTGGGCATAGGCAACGGAACCCAGATTTAT
GTAATTGATCCAGAACCGTGCCCAGATTCTGACTTCCTCCTCTGGATCCT
TGCAGCAGTTAGTTCGGGGTTGTTTTTTTATAGCTTTCTCCTCACAGCTG
TTTCTTTGAGCAAAATGCTAAAGAAAAGAAGCCCTCTTACAACAGGGGTC
(SEQ ID NO:8)

hCTLA-4 with a Y201A point Mutation and Mouse Transmembrane Domain Mouse transmembrane domain with A168V, S172L and L181V Mutations

MACLGFQRHKAQLNLATRTWPCTLLFFLLFIPVFCKAMHVAQPAVVLASS
RGIASFVCEYASPGKATEVRVTVLRQADSQVTEVCAATYMMGNELTFLDD
SICTGTSSGNQVNLTIQGLRAMDTGLYICKVELMYPPPYYLGIGNGTQIY
VIDPEPCPDSDFLLWILVAVSLGLFFYSFL(SEQ ID NO:9)

ATGGCTTGCCTTGGATTTCAGCGGCACAAGGCTCAGCTGAACCTGGCTAC
CAGGACCTGGCCCTGCACTCTCCTGTTTTTTCTTCTCTTCATCCCTGTCT
TCTGCAAAGCAATGCACGTGGCCCAGCCTGCTGTGGTACTGGCCAGCAGC
CGAGGCATCGCCAGCTTTGTGTGTGAGTATGCATCTCCAGGCAAAGCCAC
TGAGGTCCGGGTGACAGTGCTTCGGCAGGCTGACAGCCAGGTGACTGAAG
TCTGTGCGGCAACCTACATGATGGGGAATGAGTTGACCTTCCTAGATGAT
TCCATCTGCACGGGCACCTCCAGTGGAAATCAAGTGAACCTCACTATCCA
AGGACTGAGGGCCATGGACACGGGACTCTACATCTGCAAGGTGGAGCTCA
TGTACCCACCGCCATACTACCTGGGCATAGGCAACGGAACCCAGATTTAT
GTAATTGATCCAGAACCGTGCCCAGATTCTGACTTCCTCCTCTGGATCCT
TGTCGCAGTTAGTTTGGGGTTGTTTTTTTATAGCTTTCTCGTCACAGCTG
TTTCTTTGAGCAAAATGCTAAAGAAAAGAAGCCCTCTTACAACAGGGGTC
GCTGTGAAAATGCCCCCAACAGAGCCAGAATGTGAAAAGCAATTTCAGCC
TTATTTTATTCCCATCAAT(SEQ ID NO: 10)

hCTLA-4 with Mouse Transmembrane Domain no C Terminus Mouse transmembrane domain with A168, S172L and L181V Mutations

MACLGFQRHKAQLNLATRTWPCTLLFFLLFIPVFCKAMHVAQPAVVLASS
RGIASFVCEYASPGKATEVRVTVLRQADSQVTEVCAATYMMGNELTFLDD
SICTGTSSGNQVNLTIQGLRAMDTGLYICKVELMYPPPYYLGIGNGTQIY
VIDPEPCPDSDFLLWILVAVSLGLFFYSFL(SEQ ID NO:11)

ATGGCTTGCCTTGGATTTCAGCGGCACAAGGCTCAGCTGAACCTGGCTAC
CAGGACCTGGCCCTGCACTCTCCTGTTTTTTCTTCTCTTCATCCCTGTCT
TCTGCAAAGCAATGCACGTGGCCCAGCCTGCTGTGGTACTGGCCAGCAGC
CGAGGCATCGCCAGCTTTGTGTGTGAGTATGCATCTCCAGGCAAAGCCAC
TGAGGTCCGGGTGACAGTGCTTCGGCAGGCTGACAGCCAGGTGACTGAAG
TCTGTGCGGCAACCTACATGATGGGGAATGAGTTGACCTTCCTAGATGAT
TCCATCTGCACGGGCACCTCCAGTGGAAATCAAGTGAACCTCACTATCCA
AGGACTGAGGGCCATGGACACGGGACTCTACATCTGCAAGGTGGAGCTCA
TGTACCCACCGCCATACTACCTGGGCATAGGCAACGGAACCCAGATTTAT
GTAATTGATCCAGAACCGTGCCCAGATTCTGACTTCCTCCTCTGGATCCT
TGTCGCAGTTAGTTTGGGGTTGTTTTTTTATAGCTTTCTCGTCACAGCTG
TTTCTTTGAGCAAAATGCTAAAGAAAAGAAGCCCTCTTACAACAGGGGTC
(SEQ ID NO: 12)

hPDL-1 D276H + K280N Mutation

MRIFAVFIFMTYWHLLNAFTVTVPKDLYVVEYGSNMTIECKFPVEKQLDL
AALIVYWEMEDKNIIQFVHGEEDLKVQHSSYRQRARLLKDQLSLGNAALQ
ITDVKLQDAGVYRCMISYGGADYKRITVKVNAPYNKINQRILVVDPVTSE
HELTCQAEGYPKAEVIWTSSDHQVLSGKTTTTNSKREEKLFNVTSTLRIN
TTTNEIFYCTFRRLDPEENHTAELVIPELPLAHPPNERTHLVILGAILLC
LGVALTFIFRLRKGRMMDVKKCGIQHTNSNKQSDTHLEET(SEQ ID NO
:13)

ATGAGGATATTTGCTGTCTTTATATTCATGACCTACTGGCATTTGCTGAA
CGCATTTACTGTCACGGTTCCCAAGGACCTATATGTGGTAGAGTATGGTA
GCAATATGACAATTGAATGCAAATTCCCAGTAGAAAAACAATTAGACCTG
GCTGCACTAATTGTCTATTGGGAAATGGAGGATAAGAACATTATTCAATT
TGTGCATGGAGAGGAAGACCTGAAGGTTCAGCATAGTAGCTACAGACAGA
GGGCCCGGCTGTTGAAGGACCAGCTCTCCCTGGGAAATGCTGCACTTCAG
ATCACAGATGTGAAATTGCAGGATGCAGGGGTGTACCGCTGCATGATCAG
CTATGGTGGTGCCGACTACAAGCGAATTACTGTGAAAGTCAATGCCCCAT
ACAACAAAATCAACCAAAGAATTTTGGTTGTGGATCCAGTCACCTCTGAA
CATGAACTGACATGTCAGGCTGAGGGCTACCCCAAGGCCGAAGTCATCTG
GACAAGCAGTGACCATCAAGTCCTGAGTGGTAAGACCACCACCACCAATT
CCAAGAGAGAGGAGAAGCTTTTCAATGTGACCAGCACACTGAGAATCAAC
ACAACAACTAATGAGATTTTCTACTGCACTTTTAGGAGATTAGATCCTGA
GGAAAACCATACAGCTGAATTGGTCATCCCAGAACTACCTCTGGCACATC
CTCCAAATGAAAGGACTCACTTGGTAATTCTGGGAGCCATCTTATTATGC
CTTGGTGTAGCACTGACATTCATCTTCCGTTTAAGAAAAGGGAGAATGAT
GGATGTGAAAAAATGTGGCATCCAACATACAAACTCAAACAAGCAAAGTG
ATACACATTTGGAGGAGACG(SEQ ID NO:14)

What is claimed is:

1. A method of inducing immune tolerance to an agent in an individual, the method comprising administering an effective amount of an EV to the individual in conjunction with administering the agent to the individual, wherein EV comprises a lipid bilayer comprising one or more immunosuppressive molecules.

2. The method of claim 2, wherein the one or more immunosuppressive molecules comprise one or more immune checkpoint proteins.

3. The method of claim 1 or 2, wherein the one or more immunosuppressive molecules comprise one or more of CTLA4, B7-1, B7-2, PD-1, PD-L1, PD-L2, CD28, VISTA, TIM-3, GAL9, TIGIT, CD155, LAG3, VISTA, BTLA or HVEM.

4. The method of claim 1 or 2, wherein the one or more immunosuppressive molecules targets CD40 or CD40L.

5. The method of claim 4, wherein the immunosuppressive molecule is an antibody that binds CD40 or CD40L.

6. The method of any one of claims 1-5, wherein the lipid bilayer comprises two or more, three or more, or four or more different immunosuppressive molecules; or comprises two or more, three or more, or four or more different checkpoint proteins.

7. The method of any one of claims 1-6, wherein the lipid bilayer comprises CTLA4 and PD-L1; CTLA and PD-L2; CTLA-4 and VISTA; PD-L1 and PD-L2; PD-L1 and VISTA; PD-L2 and VISTA; CTLA4 and PD-L1 and PD-L2; CTLA4 and PD-L1 and VISTA; CTLA4 and PD-L2 and VISTA; PD-L1 and PD-L2 and VISTA; or CTLA4 and PD-L1 and PD-L1 and VISTA.

8. The method of any one of claims 1-7, wherein one or more of the immunosuppressive molecules comprises a transmembrane domain.

9. The method of claim 8, wherein the transmembrane domain is a PDGFR transmembrane domain, an EGFR transmembrane domain or a murine CTLA4 transmembrane domain.

10. The method of any one of claims 1-9, wherein the lipid bilayer further comprises a targeting molecule.

11. The method of claim 10, wherein the targeting molecule confers cell- or tissue-specificity to the EV.

12. The method of claim 10 or 11, wherein the targeting molecule confers specificity of the method to the liver, spleen, and/or thymus.

13. The method of claim 10 or 11, wherein the targeting molecule targets MHC class I or MHC class II mismatches between donor tissue and the individual.

14. The method of any one of claims 10-13, wherein the targeting molecule is an antibody.

15. The method of any one of claims 10-14, wherein the one or more targeting molecules comprises a transmembrane domain.

16. The method of any one of claims 1-15, wherein the EV is produced from a producer cell engineered to express the one or more immunosuppressive molecules.

17. The method of any one of claims 1-16, wherein the EV is produced from a producer cell engineered to express the one or more immunosuppressive molecules.

18. The method of any one of claims 1-17, wherein the EV is produced from a producer cell engineered to express one or more of CTLA4, B7-1, B7-2, PD-1, PD-L1, PD-L2, CD28, VISTA, TIM-3, GAL9, TIGIT, CD155, LAG3, VISTA, BTLA, HVEM, an anti-CD40 antibody or an anti-CD40L antibody.

19. The method of claim 18, wherein the producer cell is a human embryonic kidney 293 (HEK 293) cell, HeLa cell, or a Per.C6 cell.

20. The method of any one of claims 1-19, wherein the EV is produced from a producer cell which contains no additional heterologous molecules other than the one or more immunosuppressive molecules and targeting molecules.

21. The method of any one of claims 1-20, wherein the EV contains no additional molecules heterologous to the cell from which it was derived other than the one or more immunosuppressive molecules and targeting molecules.

22. The method of any one of claims 1-21, wherein the EV is administered to the individual before, at the same time, or after administration of the agent.

23. The method of any one of claims 1-22, wherein the EV is administered to the individual at the same time as administration of the agent.

24. The method of any one of claims 1-23, wherein the EV and the agent are in different formulations.

25. The method of any one of claims 1-24, wherein the EV and the agent are in the same formulation.

26. The method of claim 25, wherein the agent associates with the EV.

27. The method of claim 25 or 26, wherein the agent associates with the exterior surface of the EV.

28. The method of any one of claims 25-27, wherein the stimulation of immune tolerance facilitates repeat administration of the agent to the individual.

29. The method of claim 28, wherein the repeat administration comprises more than about 2 administrations, 3 administrations, 4 administrations, 5 administrations, 6 administrations, 7 administrations, 8 administrations, 9 administrations, or 10 administrations of the agent.

30. The method of any one of claims 1-29, wherein the agent is a therapeutic agent.

31. The method of any one of claims 1-30, wherein the agent is a polypeptide, a nucleic acid, a polypeptide-nucleic acid complex, a viral vector, a liposome, a cell, or transplanted cells or tissue.

32. The method of claim 31, wherein the agent is a therapeutic polypeptide.

33. The method of claim 32, wherein the therapeutic polypeptide is an enzyme, a hormone, an antibody, an antibody fragment, a clotting factor, a growth factor, a receptor, or a functional derivative thereof.

34. The method of claim 32 or 33, wherein the therapeutic polypeptide is Factor VIII, Factor IX, myotubularin, survival motor neuron protein (SMN), retinoid isomerohydrolase (RPE65), NADH-ubiquinone oxidoreductase chain 4, Choroideremia protein (CHM), huntingtin, alpha-galactosidase A, acid beta-glucosidase, alpha-glucosidase, ornithine transcarbomylase, argininosuccinate synthetase, β-globin, γ-globin, phenylalanine hydroxylase, adrenoleukodystrophy protein (ALD), dystrophin, a truncated dystrophin, Niemann Pick C protein (NPC-1), an anti-VEGF agent, or a functional variant thereof.

35. The method of claim 31, wherein the agent is a nucleic acid encoding a therapeutic polypeptide or a therapeutic nucleic acid.

36. The method of claim 35, wherein the nucleic acid encodes Factor VIII, Factor IX, myotubularin, survival motor neuron protein (SMN), retinoid isomerohydrolase (RPE65), NADH-ubiquinone oxidoreductase chain 4, Choroideremia protein (CHM), huntingtin, alpha-galactosidase A, acid beta-glucosidase, alpha-glucosidase, ornithine transcarbomylase, argininosuccinate synthetase, β-globin, γ-globin, phenylalanine hydroxylase, or adrenoleukodystrophy protein (ALD).

37. The method of claim 35, wherein the therapeutic nucleic acid is a siRNA, miRNA, shRNA, antisense RNA, RNAzyme, or DNAzyme.

38. The method of claim 37, wherein the nucleic acid encodes one or more gene editing products.

39. The method of claim 31, wherein the polypeptide-nucleic acid complex is a gene editing complex.

40. The method of claim 31, wherein the agent is a viral vector or a capsid protein thereof.

41. The method of claim 40, wherein the viral vector is an adeno-associated viral (AAV) vector, a lentiviral vector, an adenoviral vector, a herpes simplex viral vector or a baculovirus vector.

42. The method of claim 31, wherein the agent is a cell used in cell therapy.

43. The method of claim 42, wherein the cell is a stem cell, an induced pluripotent cell (iPS), or a differentiated cell.

44. The method of claim 42 or 43, wherein the cell is a pluripotent cell or a multipotent cell.

45. The method of claim 43 or 44, wherein the cell is an embryonic stem cell or an adult stem cell.

46. The method of claim 45, wherein the cell is a hematopoietic stem cell, a liver stem cell, a muscle stem cell, a cardiomyocyte stem cell, a neural stem cell, a bone stem cell, a mesenchymal stem cell, or an adipose stem cell.

47. The method of claim 42 or 43, wherein the cell is a blood cell, a hepatocyte, a myocyte, a cardiomyocyte, a pancreatic cell, an islet cell, an ocular cell, a neural cell, an astrocyte, an oligodendrocyte, an inner ear hair cell, a chondrocyte, or an osteoblast.

48. The method of any one of claims 42-47, wherein the cell is allogeneic to the individual.

49. The method of any one claims 1-48, wherein the individual is a human.

50. A method of treating a disease or disorder in an individual, the method comprising administering an effective amount of an EV to the individual, wherein EV comprises a lipid bilayer comprising one or more immunosuppressive molecules.

51. The method of claim 50, wherein the disease or disorder is an autoimmune disease or disorder.

52. The method of claim 50, wherein the EV is administered in conjunction with a tissue transplant or cell engraftment.

53. A method of treating a disease or disorder in an individual, the method comprising administering an effective amount of an EV to the individual in conjunction with administering an agent to the individual, wherein EV comprises a lipid bilayer comprising one or more immunosuppressive molecules, and wherein the agent treats the disease or disorder.

54. The method of any one of claims 50-53, wherein the one or more immunosuppressive molecules comprise one or more immune checkpoint proteins.

55. The method of any one of claims 50-54, wherein the one or more immunosuppressive molecules comprise one or more of CTLA4, B7-1, B7-2, PD-1, PD-L1, PD-L2, CD28, VISTA, TIM-3, GAL9, TIGIT, CD155, LAG3, VISTA, BTLA or HVEM.

56. The method of any one of claims 50-54, wherein the one or more immunosuppressive molecules targets CD40 or CD40L.

57. The method of claim 56, wherein the immunosuppressive molecule is an antibody that binds CD40 or CD40L.

58. The method of any one of claims 50-57, wherein the lipid bilayer comprises two or more, three or more, or four or more different immunosuppressive molecules; or comprises two or more, three or more, or four or more different checkpoint proteins.

59. The method of any one of claims 50-58, wherein the lipid bilayer comprises CTLA4 and PD-L1; CTLA and PD-L2; CTLA-4 and VISTA; PD-L1 and PD-L2; PD-L1 and VISTA; PD-L2 and VISTA; CTLA4 and PD-L1 and PD-L2; CTLA4 and PD-L1 and VISTA; CTLA4 and PD-L2 and VISTA; PD-L1 and PD-L2 and VISTA; or CTLA4 and PD-L1 and PD-L1 and VISTA.

60. The method of any one of claims 50-59, wherein one or more of the immunosuppressive molecules comprises a transmembrane domain.

61. The method of claim 60, wherein the transmembrane domain is a PDGFR transmembrane domain, an EGFR transmembrane domain or a murine CTLA4 transmembrane domain.

62. The method of any one of claims 50-61, wherein the lipid bilayer further comprises a targeting molecule.

63. The method of claim 62, wherein the targeting molecule confers cell- or tissue-specificity to the EV.

64. The method of claim 62 or 63, wherein the targeting molecule confers specificity of the method to the liver, spleen, and/or thymus.

65. The method of claim 62 or 63, wherein the targeting molecule targets MHC class I or MHC class II mismatches between donor tissue and the individual.

66. The method of any one of claims 62-65, wherein the targeting molecule is an antibody.

67. The method of any one of claims 62-66, wherein the one or more targeting molecules comprises a transmembrane domain.

68. The method of any one of claims 50-67, wherein the EV is produced from a producer cell engineered to express the one or more immunosuppressive molecules.

69. The method of any one of claims 50-68, wherein the EV is produced from a producer cell engineered to express the one or more immunosuppressive molecules.

70. The method of any one of claims 50-69, wherein the EV is produced from a producer cell engineered to express one or more of CTLA4, B7-1, B7-2, PD-1, PD-L1, PD-L2, CD28, VISTA, TIM-3, GAL9, TIGIT, CD155, LAG3, VISTA, BTLA, HVEM, an anti-CD40 antibody or an anti-CD40L antibody.

71. The method of any one of claims 50-70, wherein the producer cell is a human embryonic kidney 293 (HEK 293) cell, HeLa cell, or a Per.C6 cell.

72. The method of any one of claims 50-71, wherein the EV is produced from a producer cell which contains no additional heterologous molecules other than the one or more immunosuppressive molecules and targeting molecules.

73. The method of any one of claims 50-72, wherein the EV contains no additional molecules heterologous to the cell from which it was derived other than the one or more immunosuppressive molecules and targeting molecules.

74. The method of claim 50 or 73, wherein the EV is administered to the individual before, at the same time, or after administration of the agent.

75. The method of any one of claims 52-74, wherein the EV is administered to the individual at the same time as administration of the agent.

76. The method of any one of claims 52-75, wherein the EV and the agent are in different formulations.

77. The method of any one of claims 52-75, wherein the EV and the agent are in the same formulation.

78. The method of claim 77, wherein the agent associates with the EV.

79. The method of claim 77 or 78, wherein the agent associates with the exterior surface of the EV.

80. The method of any one of claims 52-79, wherein the stimulation of immune tolerance facilitates repeat administration of the agent to the individual.

81. The method of claim 80, wherein the repeat administration comprises more than about 2 administrations, 3 administrations, 4 administrations, 5 administrations, 6 administrations, 7 administrations, 8 administrations, 9 administrations, or 10 administrations of the agent.

82. The method of any one of claims 52-81, wherein the agent is a therapeutic agent.

83. The method of any one of claims 52-82, wherein the agent is a polypeptide, a nucleic acid, a polypeptide-nucleic acid complex, a viral vector, a liposome, or transplanted cells or tissue.

84. The method of claim 83, wherein the agent is a therapeutic polypeptide.

85. The method of claim 84, wherein the therapeutic polypeptide is an enzyme, a hormone, an antibody, an antibody fragment, a clotting factor, a growth factor, a receptor, or a functional derivative thereof.

86. The method of claim 84 or 85, wherein the therapeutic polypeptide is Factor VIII, Factor IX, myotubularin, survival motor neuron protein (SMN), retinoid isomerohydrolase (RPE65), NADH-ubiquinone oxidoreductase chain 4, Choroideremia protein (CHM), huntingtin, alpha-galactosidase A, acid beta-glucosidase, alpha-glucosidase, ornithine transcarbomylase, argininosuccinate synthetase, β-globin, γ-globin, phenylalanine hydroxylase, or adrenoleukodystrophy protein (ALD).

87. The method of claim 83, wherein the agent is a nucleic acid encoding a therapeutic polypeptide or a therapeutic nucleic acid.

88. The method of claim 87, wherein the nucleic acid encodes Factor VIII, Factor IX, myotubularin, survival motor neuron protein (SMN), retinoid isomerohydrolase (RPE65), NADH-ubiquinone oxidoreductase chain 4, Choroideremia protein (CHM), huntingtin, alpha-galactosidase A, acid beta-glucosidase, alpha-glucosidase, ornithine transcarbomylase, argininosuccinate synthetase, β-globin, γ-globin, phenylalanine hydroxylase, or adrenoleukodystrophy protein (ALD).

89. The method of claim 88, wherein the therapeutic nucleic acid is a siRNA, miRNA, shRNA, antisense RNA, RNAzyme, or DNAzyme.

90. The method of claim 83, wherein the nucleic acid encodes one or more gene editing products.

91. The method of claim 90, wherein the polypeptide-nucleic acid complex is a gene editing complex.

92. The method of claim 83, wherein the agent is a viral vector or a capsid protein thereof.

93. The method of claim 92, wherein the viral vector is an adeno-associated viral (AAV) vector, a lentiviral vector, an adenoviral vector, a herpes simplex viral vector or a baculovirus.

94. The method of claim 83, wherein the agent is a cell used in cell therapy.

95. The method of claim 94, wherein the cell is a stem cell, an induced pluripotent cell (iPS), or a differentiated cell.

96. The method of claim 94 or 95, wherein the cell is a pluripotent cell or a multipotent cell.

97. The method of claim 95 or 96, wherein the cell is an embryonic stem cell or an adult stem cell.

98. The method of claim 97, wherein the cell is a hematopoietic stem cell, a liver stem cell, a muscle stem cell, a cardiomyocyte stem cell, a neural stem cell, a bone stem cell, a mesenchymal stem cell, or an adipose stem cell.

99. The method of claim 94 or 95, wherein the cell is a blood cell, a hepatocyte, a myocyte, a cardiomyocyte, a pancreatic cell, an islet cell, an ocular cell, a neural cell, an astrocyte, an oligodendrocyte, an inner ear hair cell, a chondrocyte, or an osteoblast.

100. The method of any one of claims 94-99, wherein the cell is allogeneic to the individual.

101. The method of any one claims 50-100, wherein the individual is a human.

102. A composition comprising an extracellular vesicle (EV) and one or more pharmaceutically acceptable excipients for inducing immune tolerance to an agent in an individual, wherein EV comprises a lipid bilayer comprising one or more immunosuppressive molecules, and a therapeutic agent.

103. The composition of claim 102, wherein the agent is a polypeptide, a nucleic acid, a polypeptide-nucleic acid complex, a viral vector, a liposome, a cell, or transplanted cells or tissue.

104. The composition of claim 102 or 103, wherein the agent associates with the EV.

105. The composition of any one of claims 102-104, wherein the agent associates with the exterior surface of the EV.

106. The composition of any one of claims 102-105, wherein the one or more immunosuppressive molecules comprise one or more immune checkpoint proteins.

107. The composition of any one of claims 102-106, wherein the one or more immunosuppressive molecules comprise one or more of CTLA4, B7-1, B7-2, PD-1, PD-L1, PD-L2, CD28, VISTA, TIM-3, GAL9, TIGIT, CD155, LAG3, VISTA, BTLA, or HVEM.

108. The composition of any one of claims 102-107, wherein the one or more immunosuppressive molecules targets CD40 or CD40L.

109. The composition of claim 108, wherein the immunosuppressive molecule is an antibody that binds CD40 or CD40L.

110. The composition of any one of claims 102-109, wherein the lipid bilayer comprises two or more, three or more, or four or more different immunosuppressive molecules; or comprises two or more, three or more, or four or more different checkpoint proteins.

111. The composition of any one of claims 102-110, wherein the lipid bilayer comprises CTLA4 and PD-L1; CTLA and PD-L2; CTLA-4 and VISTA; PD-L1 and PD-L2; PD-L1 and VISTA; PD-L2 and VISTA; CTLA4 and PD-L1 and PD-L2; CTLA4 and PD-L1 and VISTA; CTLA4 and PD-L2 and VISTA; PD-L1 and PD-L2 and VISTA; or CTLA4 and PD-L1 and PD-L1 and VISTA.

112. The composition of any one of claims 102-111, wherein one or more of the immunosuppressive molecules comprises a transmembrane domain.

113. The composition of claim 112, wherein the transmembrane domain is a PDGFR transmembrane domain, an EGFR transmembrane domain or a murine CTLA4 transmembrane domain.

114. The composition of any one of claims 102-113, wherein the lipid bilayer further comprises a targeting molecule.

115. The composition of claim 114, wherein the targeting molecule confers cell- or tissue-specificity to the EV.

116. The composition of claim 114 or 115, wherein the targeting molecule confers specificity of the EV to the liver, spleen, and/or thymus.

117. The composition of any one of claims 114-116, wherein the targeting molecule targets MHC class I or MHC class II mismatches between donor tissue and the individual.

118. The composition of any one of claims 114-117, wherein the targeting molecule is an antibody.

119. The composition of any one of claims 114-118, wherein the one or more targeting molecules comprises a transmembrane domain.

120. The composition of any one of claims 102-119, wherein the EV is produced from a producer cell engineered to express the one or more immunosuppressive molecules.

121. The composition of any one of claims 102-120, wherein the EV is produced from a producer cell engineered to express the one or more immunosuppressive molecules.

122. The composition of any one of claims 102-121, wherein the EV is produced from a producer cell engineered to express one or more of CTLA4, B7-1, B7-2, PD-1, PD-L1, PD-L2, CD28, VISTA, TIM-3, GAL9, TIGIT, CD155, LAG3, VISTA, BTLA, HVEM, an anti-CD40 antibody or an anti-CD40L antibody.

123. The composition of any one of claims 102-122, wherein the producer cell is a human embryonic kidney 293 (HEK 293) cell, HeLa cell, or a Per.C6 cell.

124. The composition of any one of claims 102-123, wherein the EV is produced from a producer cell which contains no additional heterologous molecules other than the one or more immunosuppressive molecules and targeting molecules.

125. The composition of any one of claims 102-124, wherein the EV contains no additional molecules heterologous to the cell from which it was derived other than the one or more immunosuppressive molecules and targeting molecules.

126. A method of producing the composition of any of claims 102-125, the method comprising
a) culturing EV producer cells in vitro under conditions to generate EVs, wherein the EV producer cells comprise nucleic acids encoding one or more one or more membrane-bound immunosuppressive molecules,
b) collecting the EVs, and
c) formulating the EVs with the agent.

127. The method of claim 126, wherein the EV producer cells comprise exogenous nucleic acids encoding the membrane-bound immunosuppressive molecules.

128. The method of claim 126 or 127, wherein the membrane-bound immunosuppressive molecules comprise one or more of CTLA4, B7-1, B7-2, PD-1, PD-L1, PD-L2, CD28, VISTA, TIM-3, GAL9, TIGIT, CD155, LAG3, VISTA, BTLA or HVEM.

129. The method of claim 126 or 127, wherein the one or more immunosuppressive molecules targets CD40 or CD40L.

130. The method of claim 129, wherein the immunosuppressive molecule is an antibody that binds CD40 or CD40L.

131. The method of any one of claims 126-130, wherein one or more of the immunosuppressive molecules comprises a transmembrane domain.

132. The method of claim 131, wherein the transmembrane domain is a PDGFR transmembrane domain, an EGFR transmembrane domain or a murine CTLA4 transmembrane domain.

133. The method of any one of claims 126-132, wherein the lipid bilayer further comprises a targeting molecule.

134. The method of claim 133, wherein the targeting molecule confers cell- or tissue-specificity to the EV.

135. The method of claim 133 or 134, wherein the targeting molecule confers specificity of the EV to the liver, spleen, and/or thymus.

136. The method of claim 133 or 134, wherein the targeting molecule targets MHC class I or MHC class II mismatches between donor tissue and the individual.

137. The method of any one of claims 133-136, wherein the targeting molecule is an antibody.

138. The method of any one of claims 133-137, wherein the one or more targeting molecules comprises a transmembrane domain.

139. The method of any one of claims 126-138, wherein the EV is produced from a producer cell engineered to express the one or more immunosuppressive molecules.

140. The method of any one of claims 126-139, wherein the EV is produced from a producer cell engineered to express one or more of CTLA4, B7-1, B7-2, PD-1, PD-L1, PD-L2, CD28, VISTA, TIM-3, GAL9, TIGIT, CD155, LAG3, VISTA, BTLA or HVEM.

141. The method of claim 140, wherein the producer cell is a human embryonic kidney 293 (HEK 293) cell, HeLa cell, or a Per.C6 cell.

142. The method of any one of claims 126-141, wherein the EV is produced from a producer cell which contains no additional heterologous molecules other than the one or more immunosuppressive molecules and targeting molecules.

143. The method of any one of claims 126-142, wherein the EV contains no additional molecules heterologous to the cell from which it was derived other than the one or more immunosuppressive molecules and targeting molecules.

144. A producer cell for producing an immunosuppressive EV, wherein EV comprises a lipid bilayer comprising one or more immunosuppressive molecules, wherein the one or more immunosuppressive molecules are membrane-bound.

145. The producer cell of claim 144, wherein the producer cell is engineered to express the one or more immunosuppressive molecules.

146. The producer cell of claim 144 or 145, wherein the producer cell is engineered to express one or more of CTLA4, B7-1, B7-2, PD-1, PD-L1, PD-L2, CD28, VISTA, TIM-3, GAL9, TIGIT, CD155, LAG3, VISTA, BTLA or HVEM.

147. The producer cell of claim 144 or 145, wherein the one or more immunosuppressive molecules targets CD40 or CD40L.

148. The producer cell of claim 147, wherein the immunosuppressive molecule is an antibody that binds CD40 or CD40L.

149. The producer cell of any one of claims 144-148, wherein one or more of the immunosuppressive molecules comprises a transmembrane domain.

150. The producer cell of claim 149, wherein the transmembrane domain is a PDGFR transmembrane domain, an EGFR transmembrane domain or a murine CTLA4 transmembrane domain.

151. The producer cell of any one of claims 144-150, wherein the lipid bilayer further comprises a targeting molecule.

152. The producer cell of claim 151, wherein the targeting molecule confers cell- or tissue-specificity to the EV.

153. The producer cell of claim 151 or 152, wherein the targeting molecule confers specificity of the EV to the liver, spleen, and/or thymus.

154. The producer cell of claim 152 or 153, wherein the targeting molecule targets MHC class I or MHC class II mismatches between donor tissue and the individual.

155. The producer cell of any one of claims 151-154, wherein the targeting molecule is an antibody.

156. The producer cell of any one of claims 151-155, wherein the one or more targeting molecules comprises a transmembrane domain.

157. The producer cell of any one of claims 151-156, wherein the cell comprises nucleic acid encoding the one or more immunosuppressive molecule and/or the one or more targeting molecule.

158. The producer cell of claim 157, wherein the nucleic acid encoding the one or more immunosuppressive molecule and/or the one or more targeting molecule is stably integrated into the genome of the cell.

159. The producer cell of any one of claims 144-158, wherein the producer cell is a mammalian cell.

160. The producer cell of any one of claims 144-159, wherein the producer cell is a human cell.

161. The producer cell of any one of claims 144-160, wherein the producer cell is a human embryonic kidney 293 (HEK 293) cell, HeLa cell, or a Per.C6 cell.

162. The producer cell of any one of claims 144-161, wherein the producer cell contains no additional heterologous molecules other than the one or more immunosuppressive molecules and targeting molecules.


SEQUENCE LISTING (14 sequences)

SEQ ID NO: 1 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 2 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 3 - DNA, 669 bp, Homo sapiens
atggcttgccttggatttcagcggcacaaggctcagctgaacctggctaccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgacttcctcctctggatccttgcagcagttagttcggggttgtttttttatagctttctcctcacagctgtttctttgagcaaaatgctaaagaaaagaagccctcttacaacaggggtctatgtgaaaatgcccccaacagagccagaatgtgaaaagcaatttcagccttattttattcccatcaat

SEQ ID NO: 4 - DNA, 873 bp, Homo sapiens
atgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaa

SEQ ID NO: 5 - protein, 223 aa, Artificial Sequence
  Synthetic Construct
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVAVKMPPTEPECEKQFQPYFIPIN

SEQ ID NO: 6 - DNA, 669 bp, Artificial Sequence
  Synthetic Construct
atggcttgccttggatttcagcggcacaaggctcagctgaacctggctaccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgacttcctcctctggatccttgcagcagttagttcggggttgtttttttatagctttctcctcacagctgtttctttgagcaaaatgctaaagaaaagaagccctcttacaacaggggtcgctgtgaaaatgcccccaacagagccagaatgtgaaaagcaatttcagccttattttattcccatcaat

SEQ ID NO: 7 - protein, 200 aa, Artificial Sequence
  Synthetic Construct
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGV

SEQ ID NO: 8 - DNA, 600 bp, Artificial Sequence
  Synthetic Construct
atggcttgccttggatttcagcggcacaaggctcagctgaacctggctaccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgacttcctcctctggatccttgcagcagttagttcggggttgtttttttatagctttctcctcacagctgtttctttgagcaaaatgctaaagaaaagaagccctcttacaacaggggtc

SEQ ID NO: 9 - protein, 180 aa, Artificial Sequence
  Synthetic Construct
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILVAVSLGLFFYSFL

SEQ ID NO: 10 - DNA, 669 bp, Artificial Sequence
  Synthetic Construct
atggcttgccttggatttcagcggcacaaggctcagctgaacctggctaccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgacttcctcctctggatccttgtcgcagttagtttggggttgtttttttatagctttctcgtcacagctgtttctttgagcaaaatgctaaagaaaagaagccctcttacaacaggggtcgctgtgaaaatgcccccaacagagccagaatgtgaaaagcaatttcagccttattttattcccatcaat

SEQ ID NO: 11 - protein, 180 aa, Artificial Sequence; identical to SEQ ID NO: 9
  Synthetic Construct

SEQ ID NO: 12 - DNA, 600 bp, Artificial Sequence
  Synthetic Construct
atggcttgccttggatttcagcggcacaaggctcagctgaacctggctaccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgacttcctcctctggatccttgtcgcagttagtttggggttgtttttttatagctttctcgtcacagctgtttctttgagcaaaatgctaaagaaaagaagccctcttacaacaggggtc

SEQ ID NO: 13 - protein, 290 aa, Artificial Sequence
  Synthetic Construct
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQHTNSNKQSDTHLEET

SEQ ID NO: 14 - DNA, 870 bp, Artificial Sequence
  Synthetic Construct
atgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaacatacaaactcaaacaagcaaagtgatacacatttggaggagacg